SEQUENCE LISTING (39 sequences)

SEQ ID NO: 1 - protein, 107 aa, synthetic construct
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK

SEQ ID NO: 2 - protein, 126 aa, synthetic construct
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS

SEQ ID NO: 3 - protein, 107 aa, synthetic construct [sequence omitted]

SEQ ID NO: 4 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 5 - protein, 126 aa, synthetic construct
QVQLVQSGAEVKKPGASVKVSCKASGYTFTGKYMHWVRQAPGQGLEWMGWINPDSGGTNYAQKFQGRVTMTRDTSIATAYMELNRLRSDDTAVYYCARDQPLGYCTNGVCSYFDYWGQGTLVTVSS

SEQ ID NO: 6 - protein, 126 aa, synthetic construct
QVQLVQSGAEVKKPGASVKVSCKASGYTFTGKYMHWVRQAPGQGLEWMGWINPDSGGTNYAQKFQGRVTMTRDTSIPTAYMELNRLRSDDTAVYYCARDQPLGYCTNGVCSYFDYWGQGTLVTVSS

SEQ ID NO: 7 - protein, 126 aa, synthetic construct
QVQLVQSGAEVKKPGASVKVSCKASGYTFTGKYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS

SEQ ID NO: 8 - protein, 126 aa, synthetic construct
QVQLVQSGAEVKKPGASVKVSCKASGYTFTGRYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS

SEQ ID NO: 9 - protein, 6 aa, synthetic construct
QGIYSW

SEQ ID NO: 10 - protein, 9 aa, synthetic construct
QQANIFPLT

SEQ ID NO: 11 - protein, 8 aa, synthetic construct
  VARIANT 7: note = X is lysine (Lys, K) or arginine (Arg, R)
GYTFTGXY

SEQ ID NO: 12 - protein, 8 aa, synthetic construct
INPDSGGT

SEQ ID NO: 13 - protein, 19 aa, synthetic construct
ARDQPLGYCTNGVCSYFDY

SEQ ID NO: 14 - protein, 26 aa, synthetic construct [sequence omitted]

SEQ ID NO: 15 - protein, 17 aa, synthetic construct
LAWYQQKPGKAPNLLIY

SEQ ID NO: 16 - protein, 36 aa, synthetic construct
TLQSGVPSRFSGSGSGTDFTLTISSLQPEDFATYYC

SEQ ID NO: 17 - protein, 10 aa, synthetic construct
FGGGTKVEIK

SEQ ID NO: 18 - protein, 25 aa, synthetic construct [sequence omitted]

SEQ ID NO: 19 - protein, 17 aa, synthetic construct
MHWVRQAPGQGLEWMGW

SEQ ID NO: 20 - protein, 38 aa, synthetic construct
  VARIANT 19: note = X is alanine (Ala, A) or proline (Pro, P)
NYAQKFQGRVTMTRDTSIXTAYMELNRLRSDDTAVYYC

SEQ ID NO: 21 - protein, 11 aa, synthetic construct
WGQGTLVTVSS

SEQ ID NO: 22 - protein, 17 aa, synthetic construct
EDEPGIRCYYNDDTCQY

SEQ ID NO: 23 - protein, 18 aa, synthetic construct
EVGSYYVCPVRFQYFCEE

SEQ ID NO: 24 - protein, 13 aa, synthetic construct
GGGPLGLAGSGGS

SEQ ID NO: 25 - protein, 19 aa, synthetic construct
SGRSAGGGGPLGLAGSGGS

SEQ ID NO: 26 - protein, 137 aa, synthetic construct
EDEPGIRCYYNDDTCQYGGGPLGLAGSGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK

SEQ ID NO: 27 - protein, 138 aa, synthetic construct
EVGSYYVCPVRFQYFCEEGGGPLGLAGSGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK

SEQ ID NO: 28 - protein, 143 aa, synthetic construct
EDEPGIRCYYNDDTCQYSGRSAGGGGPLGLAGSGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK

SEQ ID NO: 29 - protein, 144 aa, synthetic construct
EVGSYYVCPVRFQYFCEESGRSAGGGGPLGLAGSGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK

SEQ ID NO: 30 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 31 - protein, 11 aa, synthetic construct
RASQGIYSWLA

SEQ ID NO: 32 - protein, 7 aa, synthetic construct
TASTLQS

SEQ ID NO: 33 - protein, 5 aa, synthetic construct
  VARIANT 2: note = X is lysine (Lys, K) or arginine (Arg, R)
GXYMH

SEQ ID NO: 34 - protein, 17 aa, synthetic construct
WINPDSGGTNYAQKFQG

SEQ ID NO: 35 - protein, 17 aa, synthetic construct
DQPLGYCTNGVCSYFDY

SEQ ID NO: 36 - protein, 7 aa, synthetic construct
  VARIANT 7: note = X is lysine (Lys, K) or arginine (Arg, R)
GYTFTGX

SEQ ID NO: 37 - protein, 6 aa, synthetic construct
NPDSGG

SEQ ID NO: 38 - protein, 6 aa, synthetic construct
PLGLAG

SEQ ID NO: 39 - protein, 5 aa, synthetic construct
SGRSA

(19) United States
(12) Patent Application Publication
(10) Pub. No.: US 2024/0279350 A1
(43) Pub. Date: Aug. 22, 2024

(54) CD40-Binding Proteins

(71) Applicant: Sanofi, Paris (FR)

(72) Inventors: Margot Cucchetti, Paris (FR); Fangyong Du, San Diego, CA (US); Yan Li, San Diego, CA (US); Guizhong Liu, San Diego, CA (US); Peter Peizhi Luo, San Diego, CA (US); Sunghae Park, Cambridge, MA (US); Yu Qiu, Cambridge, MA (US); Ingrid Sassoon, Paris (FR)

(21) Appl. No.: 18/444,128

(22) Filed: Feb. 16, 2024

Related U.S. Application Data
(63) Continuation of application No. PCT/CN2023/088096, filed on Apr. 13, 2023.

(30) Foreign Application Priority Data
Feb. 16, 2023 (EP) 23315032.5

(51) Int. Cl. C07K 16/28; A61K 39/00; C07K 1/06

(52) U.S. Cl. CPC C07K 16/2878; C07K 1/061; A61K 39/00

(57) ABSTRACT

Provided herein are anti-cluster of differentiation 40 (CD40) monoclonal antibodies, as well as conditionally-active variants thereof; and therapeutic and diagnostic methods of using said anti-CD40 monoclonal antibodies.

### CROSS-REFERENCE TO RELATED APPLICATION

This application claims the benefit of priority to European Application No. 23315032.5, filed Feb. 16, 2023, and claims the benefit of priority to International Application No. PCT/CN2023/088096, filed Apr. 13, 2023, the entire contents of each of which are incorporated herein by reference in their entirety.

### SEQUENCE LISTING

This application contains a Sequence Listing that has been submitted electronically as an XML file named “37488-0853001_SL_ST26.XML.” The XML file, created on Feb. 16, 2024, is 38,368 bytes in size. The material in the XML file is hereby incorporated by reference in its entirety.

### FIELD OF THE INVENTION

The subject disclosure relates to novel binding proteins that bind to CD40, and conditionally-active variants thereof. Therapeutic and diagnostic methods using those binding proteins are also provided. Such CD40-binding proteins are useful inter alia in the field of immuno-oncology.

### BACKGROUND

CD40 is a cell-surface member of the TNF (tumor necrosis factor) receptor superfamily. CD40 is broadly expressed on many immune cells, mostly antigen-presenting cells (APCs) at steady state including B-cells, monocytes, dendritic cells (DCs) and macrophages. CD40 is also expressed at varying degrees on non-immune cells, such as endothelial cells, epithelial/gland cells, hepatocytes, and fibroblasts. In blood, 15-20% of the circulating cells express CD40. Finally, tumor cells in multiple indications express CD40 at varying antigen density levels. CD40 binds its ligand CD40L, which is transiently expressed on T-cells and other non-immune cells under inflammatory conditions.

A wide spectrum of molecular and cellular processes is regulated by CD40 engagement, including the initiation and progression of cellular and humoral adaptive immunity. The clustering of CD40 on the cell membrane induces downstream recruitment of TNF receptor-associated factors (TRAFs), triggering several downstream signaling pathways: it induces macrophages to secrete proinflammatory mediators and enhances antigen-presenting capacity; it induces the maturation of dendritic cells and regulates antigen-presenting functions and immune activation; in B-cells, it helps antibody production, class-switching and affinity maturation. Upon activation, CD40 can also license dendritic cells to promote antitumor T-cell activation and re-educate macrophages to destroy tumor stroma. The CD40/CD40L axis thus plays a central role in APCs proliferation, as well as antigen presentation.

Agonistic anti-CD40 antibodies have been shown to suppress tumor growth in several mouse models and clinical trials, through activation of several cell types, including B-cells, macrophages and DCs. However, there remains a need for agonistic anti-CD40 antibodies with improved agonistic properties.

Additionally, occurrences of treatment-related adverse events (TRAEs), including cytokine release syndrome (CRS) and hepatotoxicity, have been reported after agonistic anti-CD40 antibody treatment in clinical trials. To address this issue, local intratumoral administration of agonistic anti-CD40 antibodies is being explored as an alternative of choice. This solution might however not be optimal, as it limits the dosage of agonistic anti-CD40 antibody treatment. It also suffers technical challenges, since the targeted tumor must have a sufficient size to ensure injectability with a needle, especially when the tumor lesions are poorly accessible. There remains thus also a need for agonistic anti-CD40 antibodies that can be administered systemically, with a better safety profile.

### BRIEF SUMMARY OF THE INVENTION

The subject specification provides anti-CD40 binding proteins. In exemplary embodiments, the anti-CD40 binding proteins of the disclosure preferentially target tumors.

In one aspect, provided herein is an antibody or antigen-binding fragment thereof, which specifically binds to CD40, preferably to human CD40, wherein said antibody or antigen-binding fragment thereof comprises (i) three light chain complementarity determining region (CDR) sequences set forth in SEQ ID NO: 1, and (ii) three heavy chain CDR sequences set forth in SEQ ID NO: 7 or 8.

In some embodiments, the antibody or antigen-binding fragment comprises three heavy chain CDR sequences set forth in SEQ ID NO: 7. In some embodiments, the antibody or antigen-binding fragment comprises three heavy chain CDR sequences set forth in SEQ ID NO: 8.

In some embodiments, the antibody or antigen-binding fragment thereof comprises: (i) a light chain variable region with SEQ ID NO: 1, or a light chain variable region sharing at least 70% of sequence identity over the non-CDR regions of SEQ ID NO: 1; and (ii) a heavy chain variable region with SEQ ID NO: 7 or 8, or a heavy chain variable region sharing at least 70% of sequence identity over the non-CDR regions of SEQ ID NO: 7 or 8.

In some embodiments, the antibody or antigen-binding fragment comprises (i) a light chain variable region with SEQ ID NO: 1; and (ii) a heavy chain variable region with an amino acid sequence selected from the group consisting of SEQ ID NOs: 5-8.

In another aspect, provided herein is an antibody or antigen-binding fragment thereof, which specifically binds to CD40, preferably to human CD40, wherein said antibody or antibody-binding fragment thereof comprises (i) a light chain variable region comprising the three following CDR sequences: VL-CDR1: QGIYSW (SEQ ID NO: 9); VL-CDR2: TAS; and VL-CDR3: QQANIFPLT (SEQ ID NO: 10); and (ii) a heavy chain variable region comprising the three following CDR sequences: VH-CDR1: GYTFTGX₁Y (SEQ ID NO: 11), wherein X₁ is selected from the group consisting of lysine (Lys, K) and arginine (Arg, R); VH-CDR2L: INPDSGGT (SEQ ID NO: 12); and VH-CDR3: ARDQPLGYCTNGVCSYFDY (SEQ ID NO: 13).

In some embodiments, X₁ is lysine (Lys, K). In some embodiments, X₁ is arginine (Arg, R).

In some embodiments, the light chain variable region further comprises the four following framework region sequences: VL-FR1: DIQMTQSPSSVSASVGDRVTITCRAS (SEQ ID NO: 14); VL-FR2: LAWYQQKPGKAPNLLIY (SEQ ID NO: 15); VL-FR3: TLQSGVPSRFSGSGSGTDFTLTISSLQPEDFATYYC (SEQ ID NO: 16); and VL-FR4: FGGGTKVEIK (SEQ ID NO: 17).

In some embodiments, the heavy chain variable region further comprises the four following framework region sequences: VH-FR1: QVQLVQSGAEVKKPGASVKVSCKAS (SEQ ID NO: 18); VH-FR2: MHWVRQAPGQGLEWMGW (SEQ ID NO: 19); VH-FR3: NYAQKFQGRVTMTRDTSIX₂TAYMELNRLRSDDTAVYYC (SEQ ID NO: 20), wherein X₂ is selected from the group consisting of alanine (Ala, A) and proline (Pro, P); and VH-FR4: WGQGTLVTVSS (SEQ ID NO: 21).

In some embodiments, the antibody or antigen-binding fragment specifically binds CD40; preferably human CD40.

In some embodiments, the antibody or antigen-binding fragment thereof is coupled to at least one masking moiety.

In another aspect, provided herein is a conditionally-active antibody or antigen-binding fragment thereof capable of specifically binding to CD40, wherein said antibody or antigen binding fragment thereof comprises: (i) three light chain complementarity determining region (CDR) sequences set forth in SEQ ID NO: 1, and (ii) three heavy chain CDR sequences set forth in SEQ ID NO: 7 or 8 or 2; and further wherein the antibody or antigen binding fragment thereof is coupled to at least one masking moiety.

In some embodiments, the at least one masking moiety reduces or inhibits binding of the antibody or antigen-binding fragment thereof to CD40.

In some embodiments, the conditionally-active antibody or antigen-binding fragment comprises three heavy chain CDR sequences set forth in SEQ ID NO: 7.

In some embodiments, the conditionally-active antibody or antigen-binding fragment comprises three heavy chain CDR sequences set forth in SEQ ID NO: 8.

In some embodiments, the conditionally-active antibody or antigen-binding fragment comprises three heavy chain CDR sequences set forth in SEQ ID NO: 2.

In some embodiments, the conditionally-active antibody or antigen-binding fragment thereof comprises: (i) a light chain variable region with SEQ ID NO: 1, or a light chain variable region sharing at least 70% of sequence identity over the non CDR regions of SEQ ID NO: 1; and (ii) a heavy chain variable region with SEQ ID NO: 7 or 8 or 2, or a heavy chain variable region sharing at least 70% of sequence identity over the non CDR regions of SEQ ID NO: 7 or 8 or 2.

In some embodiments, the conditionally-active antibody or antigen-binding fragment thereof comprises: (i) a light chain variable region with SEQ ID NO: 1; and (ii) a heavy chain variable region with an amino acid sequence selected from the group consisting of SEQ ID NOs: 5, 6, 7, 8 and 2.

In some embodiments, the at least one masking moiety is coupled to the N-terminus of the light chain variable region of the antibody or antigen-binding fragment thereof.

In some embodiments, the at least one masking moiety comprises or consists of an amino acid sequence with SEQ ID NO: 22 or 23 or an amino acid sequence sharing at least 70% of sequence identity with SEQ ID NO: 22 or 23.

In some embodiments, the conditionally-active antibody or antigen-binding fragment thereof further comprises at least one cleavable linker between the at least one masking moiety and the antibody or antigen-binding fragment thereof.

In some embodiments, the at least one cleavable linker is cleavable by at least one tumor specific protease.

In some embodiments, the at least one tumor specific protease is selected from the group consisting of matrix metalloproteinase-9 (MMP-9), urokinase type plasminogen activator (uPa), matrix metalloproteinase-2 (MMP-2), matriptase, legumain, kallikrein related peptidase 3, human neutrophil elastase, proteinase 3 (Pr3), cathepsin B, and cathepsin K.

In some embodiments, the at least one tumor specific protease is MMP-9 or uPa, or a combination thereof.

In some embodiments, the at least one cleavable linker comprises or consists of an amino acid sequence with SEQ ID NO: 24 or 25 or an amino acid sequence sharing at least 70% of sequence identity with SEQ ID NO: 24 or 25.

In some embodiments, the conditionally-active antibody or antigen-binding fragment thereof is unmasked and is capable of binding to CD40 upon cleavage of the at least one cleavable linker.

In another aspect, provided herein is a conditionally-active antibody or antigen-binding fragment thereof capable of specifically binding to CD40, wherein the antibody or antigen-binding fragment thereof is coupled to at least one masking moiety.

In some embodiments, the at least one masking moiety reduces or inhibits binding of the antibody or antigen-binding fragment thereof to CD40.

In some embodiments, the conditionally-active antibody or antigen-binding fragment thereof further comprises at least one cleavable linker between the at least one masking moiety and the antibody or antigen-binding fragment thereof.

In some embodiments, the at least one cleavable linker is cleavable by at least one tumor specific protease.

In some embodiments, the at least one tumor specific protease is selected from the group consisting of matrix metalloproteinase-9 (MMP-9), urokinase-type plasminogen activator (uPa), matrix metalloproteinase-2 (MMP-2), matriptase, legumain, kallikrein-related peptidase-3, human neutrophil elastase, proteinase 3 (Pr3), cathepsin B, and cathepsin K.

In some embodiments, the at least one tumor specific protease is MMP-9 or uPa, or a combination thereof.

In some embodiments, the at least one cleavable linker comprises or consists of an amino acid sequence with SEQ ID NO: 24 or 25 or an amino acid sequence sharing at least 70% of sequence identity with SEQ ID NO: 24 or 25.

In some embodiments, the conditionally-active antibody or antigen-binding fragment thereof is unmasked and is capable of binding to CD40 upon cleavage of the at least one cleavable linker.

In another aspect, provided herein is a conditionally-active antibody or antigen-binding fragment thereof capable of specifically binding to CD40, wherein the antibody or antigen-binding fragment thereof is coupled to at least one masking moiety comprising or consisting of an amino acid sequence with SEQ ID NO: 22 or 23 or an amino acid sequence sharing at least 70% of sequence identity with SEQ ID NO: 22 or 23.

In some embodiments, the at least one masking moiety reduces or inhibits binding of the antibody or antigen-binding fragment thereof to CD40.

In some embodiments, the conditionally-active antibody or antigen-binding fragment thereof further comprises at least one cleavable linker between the at least one masking moiety and the antibody or antigen-binding fragment thereof.

In some embodiments, the at least one cleavable linker is cleavable by at least one tumor specific protease.

In some embodiments, the at least one tumor specific protease is selected from the group consisting of matrix metalloproteinase-9 (MMP-9), urokinase type plasminogen activator (uPa), matrix metalloproteinase-2 (MMP-2), matriptase, legumain, kallikrein related peptidase 3, human neutrophil elastase, proteinase 3 (Pr3), cathepsin B, and cathepsin K.

In some embodiments, the at least one tumor specific protease is MMP-9 or uPa, or a combination thereof.

In some embodiments, the at least one cleavable linker comprises or consists of an amino acid sequence with SEQ ID NO: 24 or 25 or an amino acid sequence sharing at least 70% of sequence identity with SEQ ID NO: 24 or 25.

In some embodiments, the conditionally-active antibody or antigen-binding fragment thereof is unmasked and is capable of binding to CD40 upon cleavage of the at least one cleavable linker.

In some embodiments applicable to all of the above aspects, CD40 is human CD40.

In some embodiments applicable to all of the above aspects, the antibody or antigen-binding fragment thereof or the conditionally-active antibody or antigen-binding fragment thereof has pure agonist activity. In some embodiments, pure agonist activity means that the antibody or antigen-binding fragment thereof or the conditionally-active antibody or antigen-binding fragment thereof is capable of clustering CD40 on the surface of a cell and of activating the CD40 signaling pathway in an FcγR-independent manner or independently of any other type of target-mediated crosslinking. In some embodiments, pure agonist activity means means that the antibody or antigen-binding fragment thereof or the conditionally-active antibody or antigen-binding fragment thereof is capable of activating the CD40 signaling pathway (i) in soluble conditions, and/or (ii) in the absence of a cross-linking reagent, and/or (iii) in a FcγR-independent manner, and/or (iv) in the absence of target-mediated crosslinking of CD40.

In further aspects, provided herein is a composition comprising the antibody or antigen-binding fragment thereof, or the conditionally-active antibody or antigen-binding fragment thereof, as described herein. In some embodiments, the composition is a pharmaceutical composition and further comprises a pharmaceutically acceptable carrier or excipient.

In further aspects, provided herein is a method of treating a subject in need thereof, comprising an effective amount of the antibody or antigen-binding fragment thereof, or the conditionally-active antibody or antigen-binding fragment thereof, as described herein, or the composition as described herein. In some embodiments, the subject has cancer.

In further aspects, the antibody or antigen-binding fragment thereof, or the conditionally-active antibody or antigen-binding fragment thereof, as described herein, or the composition as described herein, is for use in treating cancer.

In further aspects, the antibody or antigen-binding fragment thereof, or the conditionally-active antibody or antigen-binding fragment thereof, as described herein, or the composition as described herein, can be used in the manufacture of a medicament for treating cancer.

In further aspects, provided herein is an isolated polynucleotide encoding the antibody or antigen-binding fragment thereof, or the conditionally-active antibody or antigen-binding fragment thereof, as described herein; a vector comprising the polynucleotide as described herein; or a host cell comprising the polynucleotide as described herein.

In further aspects, provided herein is a method of manufacturing the antibody or antigen-binding fragment thereof, or the conditionally-active antibody or antigen-binding fragment thereof, as described herein, comprising expressing the polynucleotide or vector as described herein, in a cell.

In further aspects, provided herein is a method of manufacturing the antibody or antigen-binding fragment thereof, or the conditionally-active antibody or antigen-binding fragment thereof, as described herein, comprising cultivating a host cell as described herein in a culture medium, and recovering the antibody or antigen binding fragment thereof, or the conditionally-active antibody or antigen-binding fragment thereof, thereby produced by the host cell.

The summary of the disclosure described above is non-limiting and other features and advantages of the disclosed antigen-binding proteins and methods will be apparent from the following brief description of the drawings, detailed description of the disclosure, and claims.

### BRIEF DESCRIPTION OF THE FIGURES

FIGS. 1A to 1C graphically depict the affinity of the parental antibody [AbC1] and mutant antibody constructs [mAb1; mAb2; mAb3; mAb4] vs isotype control in a B-cell binding assay on human primary naive B-cells (FIGS. 1A-B) and on cynomolgus CD40-expressing HEK293 cells (FIG. 1C). MFI: mean fluorescence intensity.

FIGS. 2A and 2B graphically depict the activity of the parental antibody [AbC1] and mutant antibody constructs [mAb1; mAb2; mAb3] vs isotype control in a reporter assay in human NFkB-Luc2P/U-2 OS cells (FIG. 2A) and in rhesus CD40-expressing HEK293 cells (FIG. 2B). Results are depicted as the ratio of luciferase activity of each sample to the luciferase activity of negative serum (signal-to-noise [S/N] ratio).

FIGS. 3A to 3C depict the expression of activation markers and costimulatory molecules CD86 (FIG. 3A), CD69 (FIG. 3B) and CD267 (“TACI”; FIG. 3C), in a B-cell activation assay using various concentrations of a positive control [hexameric CD40L], an isotype control, the parental antibody [AbC1] and mutant antibody constructs [mAb1; mAb2; mAb3; mAb4]. MFI: mean fluorescence intensity.

FIGS. 4A to 4F depict the expression of the monocyte-derived dendritic cell maturation markers CD80 (FIGS. 4A-B), CD83 (FIGS. 4C-D) and CD86 (FIGS. 4E-F) in a primary monocyte-derived dendritic cell activation assay using various concentrations of a positive control [hexameric CD40L], an isotype control, the parental antibody [AbC1] and mutant antibody constructs (FIGS. 4A, 4C, 4E: [mAb1; mAb2]; FIGS. 4B, 4D, 4F: [mAb3; mAb4]). MFI: mean fluorescence intensity.

FIG. 5 depicts the results of a binding assay on Raji cells with a mutant antibody construct [mAb1], 4 masked compounds [MC1-MC4], 2 protease-activated compounds [MMP9-activated; uPa-activated] and an isotype control [isotype].

FIGS. 6A and 6B depict the results of a binding assay on U-2 OS/NFκB cells with the parental antibody [AbC1], a mutant antibody construct [mAb1], 4 masked compounds [MC1-MC4], protease-activated versions thereof [MMP9-activated; uPa-activated] and an isotype control.

FIGS. 7A and 7B depict the results of a luciferase-based reporter assay in U-2 OS/NFκB cells with the parental antibody [AbC1], a mutant antibody construct [mAb1], 4 masked compounds [MC1-MC4], protease-activated versions thereof [MMP9-activated; uPa-activated]and an isotype control. RLU: relative light unit. S/N ratio: signal-to-noise ratio.

FIG. 8 depicts the results of a primary human B-cell activation assay with a mutant antibody construct [mAb1], 4 masked compounds [MC1-MC4], 2 protease-activated compounds [MMP9-activated; uPa-activated] and an isotype control [isotype]. MFI: mean fluorescence intensity.

FIGS. 9A and 9B depict the results of a murine B-cell activation assay with the parental antibody [AbC1], a positive control [hexameric CD40L], a mutant antibody construct [mAb1], 4 masked compounds [MC1-MC4], protease-activated versions thereof [MMP9-activated] and an isotype control. FIG. 9A: expression of CD83 from activated murine B-cells; FIG. 9B: expression of CD86 from activated murine B-cells. MFI: mean fluorescence intensity.

FIGS. 10A to 10F depict activity of the parental antibody [AbC1], a positive control [hexameric CD40L], a mutant antibody construct [mAb1], 4 masked compounds [MC1-MC4], protease-activated versions thereof [MMP9-activated] and an isotype control, in a monocyte-derived dendritic cell maturation assay. FIG. 10A shows the expression of CD83 from mature monocyte-derived dendritic cells (MFI: mean fluorescence intensity); FIG. 10B shows the release of interleukin-8 [IL-8] from mature monocyte-derived dendritic cells; FIG. 10C shows donor-dependent activity expressed as the EC₅₀ for CD86 expression; FIG. 10D shows donor-dependent activity expressed as the EC₅₀ for CD83 expression; FIG. 10E shows donor-dependent activity expressed as the EC₅₀ for CD80 expression; FIG. 10F shows donor-dependent activity expressed as the EC₅₀ for interleukin-8 [IL-8] release.

### DETAILED DESCRIPTION

Before the present invention is described, it is to be understood that this disclosure is not limited to particular methods and experimental conditions described herein, as such methods and conditions may vary. It is also to be understood that the terminology used herein is for the purpose of describing particular embodiments only, and is not intended to be limiting, since the scope of the present disclosure will be limited only by the appended claims.

### Definitions

Unless defined otherwise, all technical and scientific terms used herein have the same meaning as commonly understood by one of ordinary skill in the art to which this invention belongs.

The term “about”, when used in reference to a particular recited numerical value, means that the value may vary from the recited value by no more than 5%, preferably no more than 2%, and more preferably no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice of the present disclosure, exemplary methods and materials are now described. All publications mentioned herein are incorporated herein by reference to describe in their entirety.

The term “CD40” refers to a cell-surface member of the TNFR superfamily, also named “tumor necrosis factor receptor superfamily member 5” or “TNFRSF5”. CD40 is expressed by B-cells, professional antigen-presenting cells, as well as non-immune cells and tumors. CD40 binds its ligand CD40L (also named TNFSF5), which is transiently expressed on T-cells and other non-immune cells under inflammatory conditions. Upon activation, CD40 can license dendritic cells to promote antitumor T-cell activation and re-educate macrophages to destroy tumor stroma. An exemplary amino acid sequence of CD40 is set forth in SEQ ID NO: 30, corresponding to human CD40 (hCD40) with Uniprot accession number P25942-1 (last updated 1992 May 1).

SEQ ID NO: 30
MVRLPLQCVLWGCLLTAVHPEPPTACREKQYLINSQCCSLCQPGQKLVS
DCTEFTETECLPCGESEFLDTWNRETHCHQHKYCDPNLGLRVQQKGTSE
TDTICTCEEGWHCTSEACESCVLHRSCSPGFGVKQIATGVSDTICEPCP
VGFFSNVSSAFEKCHPWTSCETKDLVVQQAGTNKTDVVCGPQDRLRALV
VIPIIFGILFAILLVLVFIKKVAKKPTNKAPHPKQEPQEINFPDDLPGS
NTAAPVQETLHGCQPVTQEDGKESRISVQERQ

The term “antibody”, as used herein, is intended to refer to immunoglobulin (Ig) molecules comprised of four polypeptide chains—two heavy (H) chains and two light (L) chains inter-connected by disulfide bonds (i.e., “full antibody molecules”), as well as multimers thereof (e.g., IgM) or antigen-binding fragments thereof. Each heavy chain is comprised of a heavy chain variable region (“HCVR” or “VH”) and a heavy chain constant region (“HCCR” or “C_{H}”; comprised of domains C_{H}1, C_{H}2 and C_{H}3). Each light chain is comprised of a light chain variable region (“LCVR or “V_{L}”) and a light chain constant region (“LCCR” or “C_{L}”). The VH and V_{L}regions can be further subdivided into regions of hypervariability, termed complementarity determining regions (CDR), interspersed with regions that are more conserved, termed framework regions (FR). Each VH and V_{L} is composed of three CDRs and four FRs, arranged from amino-terminus to carboxy-terminus in the following order: FR1, CDR1, FR2, CDR2, FR3, CDR3, FR4. In certain embodiments of the disclosure, the FRs of the antibody (or antigen-binding fragment thereof) may be identical to the human germline sequences, or may be naturally or artificially modified. An amino acid consensus sequence may be defined based on a side-by-side analysis of two or more CDRs. By default, CDRs and FRs sequences are herein given as defined by IMGT numbering unless defined otherwise.

Substitution of one or more CDR residues or omission of one or more CDRs is also possible. Antibodies have been described in the scientific literature in which one or two CDRs can be dispensed with for binding. Padlan et al. (FASEB J. 1995; 9(1):133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al., J Mol Biol. 2002; 320(2):415-428). CDR residues not contacting antigen can be identified based on previous studies (for example, residues H60-H65 in VH-CDR2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

The anti-CD40 antibodies disclosed herein may comprise one or more amino acid substitutions, insertions and/or deletions in the framework and/or CDR regions of the heavy and/or light chain variable domains as compared to the corresponding germline sequences. Such mutations can be readily ascertained by comparing the amino acid sequences disclosed herein to germline sequences available from, for example, public antibody sequence databases. The present disclosure includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present disclosure may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

The present disclosure also includes anti-CD40 antibodies comprising variants of any of the HCVR, LCVR, and/or CDR amino acid sequences disclosed herein having one or more conservative substitutions. For example, the present disclosure includes anti-CD40 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “human antibody”, as used herein, is intended to include antibodies having variable and constant regions derived from human germline immunoglobulin sequences. The human mAbs of the disclosure may include amino acid residues not encoded by human germline immunoglobulin sequences (e.g., mutations introduced by random or site-specific mutagenesis in vitro or by somatic mutation in vivo), for example in the CDRs and, in particular, in CDR3. However, the term “human antibody”, as used herein, is not intended to include mAbs in which CDR sequences derived from the germline of another mammalian species (e.g., mouse) have been grafted onto human FR sequences. The term includes antibodies recombinantly produced in a non-human mammal, or in cells of a non-human mammal. The term is not intended to include antibodies isolated from, or generated in, a human subject.

The term “recombinant”, as used herein, refers to antibodies or antigen-binding fragments thereof of the disclosure created, expressed, isolated or otherwise obtained by technologies or methods known in the art as recombinant DNA technology, which include, e.g., DNA splicing and transgenic expression. The term refers to antibodies or antigen-binding fragments thereof expressed in a non-human mammal (including transgenic non-human mammals, e.g., transgenic mice), or a cell (e.g., CHO cells) expression system, or isolated from a recombinant combinatorial human antibody library.

The term “multispecific antigen-binding molecules”, as used herein, refers to bispecific, tri-specific or multi-specific antigen-binding molecules, and antigen-binding fragments thereof. Multispecific antigen-binding molecules may be specific for different epitopes of one target polypeptide or may contain antigen-binding domains specific for epitopes of more than one target polypeptide. A multispecific antigen-binding molecule can be a single multifunctional polypeptide, or it can be a multimeric complex of two or more polypeptides that are covalently or non-covalently associated with one another. The term “multispecific antigen-binding molecules”includes antibodies or antigen-binding fragment thereof of the present disclosure that may be linked to or co-expressed with another functional molecule, e.g., another peptide or protein. For example, an antibody or antigen-binding fragment thereof can be functionally linked (e.g., by chemical coupling, genetic fusion, non-covalent association or otherwise) to one or more other molecular entities, such as a protein or fragment thereof to produce a bi-specific or a multi-specific antigen-binding molecule with a second binding specificity. According to the present disclosure, the term “multispecific antigen-binding molecules” also includes bispecific, tri-specific or multi-specific antibodies or antigen-binding fragments thereof. In certain exemplary embodiments, an antibody or antigen-binding fragments thereof of the present disclosure is functionally linked to another antibody or antigen-binding fragment thereof to produce a bispecific antibody with a second binding specificity.

The terms “specifically bind(s)”, “bind(s) specifically to”, and any declension thereof, mean that an antibody or antigen-binding fragment thereof forms a complex with an antigen that is relatively stable under physiologic conditions. Specific binding can be characterized by an equilibrium dissociation constant (denoted “K_{D}”) of at least about 1×10⁻⁸ M or less (e.g., a smaller K_{D} denotes a tighter binding). Methods for determining whether two molecules specifically bind are well-known in the art and include, without limitation, equilibrium dialysis, surface plasmon resonance, bio-layer interferometry, and the like.

The term “high affinity”, with respect to an antibody or antigen-binding fragment thereof, refers to those mAbs having a binding affinity to antigen, e.g., CD40, expressed as K_{D}, of at least 10⁻⁷ M, at least 10⁻⁸ M, at least 10⁻⁹ M, at least 10⁻¹⁰ M, or at least 10⁻¹¹ M, as measured by surface plasmon resonance, e.g., BIACORE™, bio-layer interferometry, or solution-affinity ELISA.

The term “off rate”, or “K_{off}”, refers a constant used to characterize how quickly an antibody or antigen-binding fragment thereof dissociates from its antigen, e.g., CD40. By “slow off rate”, it is meant an antibody or antigen-binding fragment thereof that dissociates from its antigen, e.g., CD40, with a rate constant of 1×10⁻³ s⁻¹ or less, or of 1×10⁻⁴ s⁻¹ or less, as determined by surface plasmon resonance, e.g., BIACORE™, or bio-layer interferometry.

The term “surface plasmon resonance”, as used herein, refers to an optical phenomenon that allows for the analysis of real-time biomolecular interactions by detection of alterations in protein concentrations within a biosensor matrix, for example using the BIACORE™ system (Pharmacia Biosensor AB, Uppsala, Sweden and Piscataway, N.J.).

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment,” as used herein, refers to one or more fragments of an antibody that retain the ability to bind to CD40.

In specific embodiments, antibody or antigen-binding fragments thereof of the disclosure may be conjugated to a moiety such a ligand or a therapeutic moiety (“immunoconjugate”), such as an antibiotic, a second anti-CD40 antibody, or an antibody to another antigen such a tumor-specific antigen, an autoimmune tissue antigen, a virally-infected cell antigen, a Fc receptor, a T-cell receptor, or a T-cell co-inhibitor, or an immunotoxin, or any other therapeutic moiety useful for treating a disease or condition including cancer, autoimmune disease or chronic viral infection.

An “isolated antibody”, as used herein, is intended to refer to an antibody that is substantially free of other antibodies (Abs) having different antigenic specificities (e.g., an isolated antibody that specifically binds CD40, or an antigen-binding fragment thereof, is substantially free of Abs that specifically bind antigens other than CD40).

The terms “activating antibody”, “enhancing antibody”, and “agonist antibody”, as used herein, are intended to refer to an antibody or antigen-binding fragment thereof whose binding to CD40 results in increasing or stimulating at least one biological activity of CD40. For example, an “activating antibody”, “enhancing antibody”, or “agonist antibody” can refer to an antibody or antigen-binding fragment thereof which mimics the action of CD40's natural ligand, CD40L (e.g., by triggering CD40 clustering) to promote the maturation of dendritic cells (DCs) and improve their antigen presentation capabilities, ultimately resulting in expansion of tumor antigen-specific cytotoxic T-cells.

The term “pure agonist”, as used herein, is intended to refer to an activating antibody or antigen-binding fragment thereof as defined hereinabove, whose binding to its antigen results in increasing or stimulating at least one biological activity of the antigen (e.g., by activating the CD40 signaling pathway) (i) in soluble conditions and/or (ii) in the absence of a cross-linking reagent and/or (iii) in an FcγR-independent manner (i.e., independently of Fcγ receptor engagement) and/or (iv) in the absence of target-mediated crosslinking of the antigen.

The term “Fc-mediated crosslinking”, as used herein, refers to the crosslinking of a protein comprising an Fc domain (e.g., an antibody) via binding of said Fc domain to an Fc-binding moiety, e.g., an anti-Fc antibody or an Fc receptor.

As used herein, the term “Fe receptor” refers to the surface receptor protein found on immune cells including B-lymphocytes, natural killer cells, macrophages, basophils, neutrophils, and mast cells, which has a binding specificity for the Fc region of an antibody. The term “Fc receptor” includes, but is not limited to, a Fcγ receptor [e.g., FcγRI (CD64), FcγRIIA (CD32), FcγRIIB (CD32), FcγRIIIA (CD16a), and FcγRIIIB (CD16b)], Fcα receptor (e.g., FcαRI or CD89) and Fcε receptor [e.g., FcεRI, and FcεRII (CD23)].

The term “target-mediated crosslinking”, as used herein, refers to the crosslinking of an antibody or antigen-binding fragment thereof, via interaction of this antibody or antigen-binding fragment thereof with, e.g., a tumor-associated antigen (TAA), an immune cell surface marker, a stromal antigen or any other target expressed in cis or trans by a tumor cell, an immune cell, and/or a normal cell.

The term “epitope” refers to an antigenic determinant that interacts with a specific antigen-binding site in the variable region of an antibody known as a “paratope”. A single antigen may have more than one epitope. Thus, different antibodies may bind to different areas on a same antigen and may have different biological effects. The term “epitope” also refers to a site on an antigen to which B- and/or T-cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids.

In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “substantial identity” or “substantially identical”, when referring to a nucleic acid or fragment thereof, indicates that, when optimally aligned with appropriate nucleotide insertions or deletions with another nucleic acid (or its complementary strand), there is nucleotide sequence identity in at least about 90%, at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or a substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity, at least 95%, 96%, 97%, 98% or 99% of sequence identity. In exemplary embodiments, residue positions, which are not identical, may differ by conservative amino acid substitutions. A “conservative amino acid substitution” is one in which an amino acid residue is substituted by another amino acid residue having a side chain (R group) with similar chemical properties (e.g., in terms of charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (Methods Mol Biol. 1994; 24:307-31), which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Exemplary conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al., Science. 1992 Jun. 5; 256(5062):1443-5, herein incorporated by reference. A “moderately conservative” replacement is any change having a non-negative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild-type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson, Methods Mol Biol. 2000; 132:185-219). Another exemplary algorithm when comparing a sequence of the disclosure to a database containing a large number of sequences from different organisms is the computer program BLAST, especially BLASTP or TBLASTN, using default parameters. See, e.g., Altschul et al., J Mol Biol. 1990 Oct. 5; 215(3):403-10 and Altschul et al., Nucleic Acids Res. 1997 Sep. 1; 25(17):3389-402, each of which is herein incorporated by reference.

In some embodiments, the antibody or antigen-binding fragment thereof disclosed herein may be conditionally-active or activatable. By “conditionally-active” or “activatable”, it is meant herein that the antibody or antigen-binding fragment thereof is capable of binding to its antigen (i.e., to be active) only under certain condition. In some exemplary embodiments, a conditionally-active antibody or antigen-binding fragment thereof comprises a masking moiety.

As used herein, the terms “mask”, “masking domain” or “masking moiety” refer to a moiety that is added to an antibody or antigen-binding fragment thereof to reduce the ability of the antibody to bind its antigen. The mask serves to prevent or reduce antigen binding by one or more CDR sequences of the antibody or antigen-binding fragment thereof. Masking moieties include, but are not limited to, autologous hinge domains, coiled-coil domains, non-antibody protein fragments, antibody fragments, affinity peptides, cross-masking antibodies, bivalent peptide-double strand DNA conjugates and the like. For a review of suitable antibody masking moieties, see Lin et al. (J Biomed Sci. 2020 Jun. 25; 27(1):76).

In certain exemplary embodiments, a masking moiety is a polypeptide that may be removed from the antibody or antigen-binding fragment thereof by cleavage of a cleavable linker connecting the masking moiety to the antibody or antigen-binding fragment thereof, thereby allowing the antibody or antigen-binding fragment thereof to bind to its target antigen. In particularly exemplary embodiments, a masking domain of an antibody or antigen-binding fragment thereof is cleaved at a tumor site, e.g., at a tumor bed or at a lymph node. The cleavable linker may a protease-cleavable linker. In certain exemplary embodiments, the cleavable linker comprises at least one substrate for a tumor-specific protease.

By the phrase “therapeutically effective amount” is meant an amount that produces the desired effect for which it is administered. The exact amount will depend on the purpose of the treatment, and will be ascertainable by one skilled in the art using known techniques (see, for example, Lloyd (1999) The Art, Science and Technology of Pharmaceutical Compounding).

The term “subject”, as used herein, refers to an animal, e.g., a mammal, in need of amelioration, prevention and/or treatment of a disease or disorder such as cancer, or a chronic viral infection. In some embodiments, the subject is a human subject in need of amelioration, prevention and/or treatment of a disease or disorder such as cancer, or a chronic viral infection.

As used herein, “anti-cancer drug” means any agent useful to treat cancer including, but not limited to, cytotoxins and agents such as antimetabolites, alkylating agents, anthracyclines, antibiotics, antimitotic agents, procarbazine, hydroxyurea, asparaginase, corticosteroids, mytotane (O,P′-(DDD)), biologics (e.g., antibodies and interferons) and radioactive agents.

As used herein, a “cytotoxin” or “cytotoxic agent”, also referred to as “chemotherapeutic agent”, means any agent that is detrimental to cells. Examples include TAXOL® (paclitaxel), temozolamide, cytochalasin B, gramicidin D, ethidium bromide, emetine, cisplatin, mitomycin, etoposide, tenoposide, vincristine, vinbiastine, coichicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

As used herein, the term “antiviral drug” refers to any drug or therapy used to treat, prevent, or ameliorate a viral infection in a host subject. The term “antiviral drug” includes, but is not limited to zidovudine, lamivudine, abacavir, ribavirin, lopinavir, efavirenz, cobicistat, tenofovir, rilpivirine, analgesics and corticosteroids. In the context of the present disclosure, the viral infections include long-term or chronic infections caused by viruses including, but not limited to, human immunodeficiency virus (HIV), hepatitis B virus (HBV), hepatitis C virus (HCV), human papilloma virus (HPV), lymphocytic choriomeningitis virus (LCMV), and simian immunodeficiency virus (SIV).

In some embodiments, the antibodies may be useful for stimulating or enhancing the immune response and/or for treating a subject suffering from cancer, or a chronic viral infection. The antibodies when administered to a subject in need thereof may reduce the chronic infection by a virus such as HIV, LCMV or HBV in the subject. They may be used to inhibit the growth of tumor cells in a subject. They may be used alone or as adjunct therapy with other therapeutic moieties or modalities known in the art for treating cancer, or viral infection.

### DETAILED DESCRIPTION OF THE INVENTION

In a first aspect, antibodies or antigen-binding fragments thereof, which specifically binds to CD40, preferably to human CD40 are provided, wherein the antibodies or antigen-binding fragments thereof comprise:
three light chain complementarity determining region (CDR) sequences set forth in SEQ ID NO: 1, and
three heavy chain CDR sequences set forth in SEQ ID NO: 7 or 8.

In some embodiments, the three heavy chain CDR sequences of the antibodies or antigen-binding fragments are as set forth in SEQ ID NO: 7. In some embodiments, the three heavy chain CDR sequences of the antibodies or antigen-binding fragments are as set forth in SEQ ID NO: 8.

In some embodiments, the antibodies or antigen-binding fragments thereof comprise the following CDR sequences (as defined by IMGT numbering):
light chain CDR1 (V_{L}-CDR1) with the amino acid sequence QGIYSW (SEQ ID NO: 9);
light chain CDR2 (V_{L}-CDR2) with the amino acid sequence TAS; and
light chain CDR3 (V_{L}-CDR3) with the amino acid sequence QQANIFPLT (SEQ ID NO: 10); and
heavy chain CDR1 (V_{H}-CDR1) with the amino acid sequence GYTFTGX₁Y (SEQ ID NO: 11), wherein X₁ is selected from the group consisting of lysine (Lys, K) and arginine (Arg, R);
heavy chain CDR2 (V_{H}-CDR2) with the amino acid sequence INPDSGGT (SEQ ID NO: 12); and
heavy chain CDR3 (V_{H}-CDR3) with the amino acid sequence ARDQPLGYCTNGVCSYFDY (SEQ ID NO: 13).

In some embodiments, X₁ in V_{H}-CDR1 is lysine (Lys, K). In some embodiments, X₁ in V_{H}-CDR1 is arginine (Arg, R).

In some embodiments, the antibodies or antigen-binding fragments thereof comprise the following CDR sequences (as defined by Kabat numbering):
light chain CDR1 (V_{L}-CDR1) with the amino acid sequence RASQGIYSWLA (SEQ ID NO: 31);
light chain CDR2 (V_{L}-CDR2) with the amino acid sequence TASTLQS (SEQ ID NO: 32); and
light chain CDR3 (V_{L}-CDR3) with the amino acid sequence QQANIFPLT (SEQ ID NO: 10); and
heavy chain CDR1 (V_{H}-CDR1) with the amino acid sequence GXiYMH (SEQ ID NO: 33), wherein X₁ is selected from the group consisting of lysine (Lys, K) and arginine (Arg, R);
heavy chain CDR2 (V_{H}-CDR2) with the amino acid sequence WINPDSGGTNYAQKFQG (SEQ ID NO: 34); and
heavy chain CDR3 (V_{H}-CDR3) with the amino acid sequence DQPLGYCTNGVCSYFDY (SEQ ID NO: 35).

In some embodiments, X₁ in V_{H}-CDR1 is lysine (Lys, K). In some embodiments, X₁ in V_{H}-CDR1 is arginine (Arg, R).

In some embodiments, the antibodies or antigen-binding fragments thereof comprise the following CDR sequences (as defined by Chothia numbering):
light chain CDR1 (V_{L}-CDR1) with the amino acid sequence RASQGIYSWLA (SEQ ID NO: 31);
light chain CDR2 (V_{L}-CDR2) with the amino acid sequence TASTLQS (SEQ ID NO: 32);
and
light chain CDR3 (V_{L}-CDR3) with the amino acid sequence QQANIFPLT (SEQ ID NO: 10);
and
heavy chain CDR1 (V_{H}-CDR1) with the amino acid sequence GYTFTGX₁ (SEQ ID NO: 36), wherein X₁ is selected from the group consisting of lysine (Lys, K) and arginine (Arg, R);
heavy chain CDR2 (V_{H}-CDR2) with the amino acid sequence NPDSGG (SEQ ID NO: 37);
and
heavy chain CDR3 (V_{H}-CDR3) with the amino acid sequence DQPLGYCTNGVCSYFDY (SEQ ID NO: 35).

In some embodiments, X₁ in V_{H}-CDR1 is lysine (Lys, K). In some embodiments, X₁ in V_{H}-CDR1 is arginine (Arg, R).

In some embodiments, the antibodies or antigen-binding fragments thereof comprise:
a light chain variable region sharing at least 70%, 75%, 80%, 85%, 90%, 95%, 96%, 97%, 98%, or 99% of sequence identity over the non-CDR regions of SEQ ID NO: 1; and
a heavy chain variable region sharing at least 70%, 75%, 80%, 85%, 90%, 95%, 96%, 97%, 98%, or 99% of sequence identity over the non-CDR regions of SEQ ID NO: 5, 6, 7, or 8.

In some embodiments, the antibodies or antigen-binding fragments thereof comprise:
a light chain variable region with SEQ ID NO: 1; and
a heavy chain variable region with SEQ ID NO: 5, 6, 7, or 8.

In some embodiments, the antibodies or antigen-binding fragments thereof comprise:
a light chain variable region sharing at least 70%, 75%, 80%, 85%, 90%, 95%, 96%, 97%, 98%, or 99% of sequence identity over the non-CDR regions of SEQ ID NO: 1; and
a heavy chain variable region sharing at least 70%, 75%, 80%, 85%, 90%, 95%, 96%, 97%, 98%, or 99% of sequence identity over the non-CDR regions of SEQ ID NO: 7 or 8.

In some embodiments, the antibodies or antigen-binding fragments thereof comprise:
a light chain variable region with SEQ ID NO: 1; and
a heavy chain variable region with SEQ ID NO: 7 or 8.

In some embodiments, the antibodies or antigen-binding fragments thereof further comprise a substitution in the third heavy chain framework region (V_{H}-FR3), at position S77 of SEQ ID NO: 7 or 8. In some embodiments, this substitution is selected from the group consisting of S77A and S77P.

In some embodiments, the antibodies or antigen-binding fragments thereof comprise:
a light chain variable region sharing at least 70%, 75%, 80%, 85%, 90%, 95%, 96%, 97%, 98%, or 99% of sequence identity over the non-CDR regions of SEQ ID NO: 1; and
a heavy chain variable region sharing at least 70%, 75%, 80%, 85%, 90%, 95%, 96%, 97%, 98%, or 99% of sequence identity over the non-CDR regions of SEQ ID NO: 5 or 6, wherein position 77 of SEQ ID NO: 5 or 6 retains an alanine (Ala, A) or a proline (Pro, P) residue.

In some embodiments, the antibodies or antigen-binding fragments thereof comprise:
a light chain variable region with SEQ ID NO: 1; and
a heavy chain variable region with SEQ ID NO: 5 or 6.

In some embodiments, the antibodies or antigen-binding fragments thereof described hereinabove bind to, in particular specifically bind to, CD40. In some embodiments, CD40 is human CD40, an exemplary amino acid sequence of which is SEQ ID NO: 30.

In some embodiments, the antibodies or antigen-binding fragments thereof is coupled to at least one masking moiety.

In a second aspect, conditionally-active antibodies or antigen-binding fragments thereof are provided.

In some embodiments, the conditionally-active antibodies or antigen-binding fragments thereof comprise:
three light chain complementarity determining region (CDR) sequences set forth in SEQ ID NO: 1, and
three heavy chain CDR sequences set forth in SEQ ID NO: 7 or 8 or 2;
and the conditionally-active antibodies or antigen-binding fragments thereof are coupled to at least one masking moiety.

In some embodiments, the at least one masking moiety reduces or inhibits binding of the antibody or antigen-binding fragment thereof to its target antigen.

In some embodiments, the three heavy chain CDR sequences of the conditionally-active antibodies or antigen-binding fragments are as set forth in SEQ ID NO: 7. In some embodiments, the three heavy chain CDR sequences of the conditionally-active antibodies or antigen-binding fragments are as set forth in SEQ ID NO: 8. In some embodiments, the three heavy chain CDR sequences of the conditionally-active antibodies or antigen-binding fragments are as set forth in SEQ ID NO: 2.

In some embodiments, the conditionally-active antibodies or antigen-binding fragments thereof comprise the following CDR sequences (as defined by IMGT numbering):
light chain CDR1 (V_{L}-CDR1) with the amino acid sequence QGIYSW (SEQ ID NO: 9);
light chain CDR2 (V_{L}-CDR2) with the amino acid sequence TAS; and
light chain CDR3 (V_{L}-CDR3) with the amino acid sequence QQANIFPLT (SEQ ID NO: 10); and
heavy chain CDR1 (V_{H}-CDR1) with the amino acid sequence GYTFTGX₁Y (SEQ ID NO: 11), wherein X₁ is selected from the group consisting of lysine (Lys, K), arginine (Arg, R) and tyrosine (Tyr, Y);
heavy chain CDR2 (V_{H}-CDR2) with the amino acid sequence INPDSGGT (SEQ ID NO: 12); and
heavy chain CDR3 (V_{H}-CDR3) with the amino acid sequence ARDQPLGYCTNGVCSYFDY (SEQ ID NO: 13).

In some embodiments, X₁ in V_{H}-CDR1 is lysine (Lys, K). In some embodiments, X₁ in V_{H}-CDR1 is arginine (Arg, R). In some embodiments, X₁ in V_{H}-CDR1 is tyrosine (Tyr, Y).

In some embodiments, the conditionally-active antibodies or antigen-binding fragments thereof comprise the following CDR sequences (as defined by Kabat numbering):
light chain CDR1 (V_{L}-CDR1) with the amino acid sequence RASQGIYSWLA (SEQ ID NO: 31);
light chain CDR2 (V_{L}-CDR2) with the amino acid sequence TASTLQS (SEQ ID NO: 32); and
light chain CDR3 (V_{L}-CDR3) with the amino acid sequence QQANIFPLT (SEQ ID NO: 10); and
heavy chain CDR1 (V_{H}-CDR1) with the amino acid sequence GX₁YMH (SEQ ID NO: 33), wherein X₁ is selected from the group consisting of lysine (Lys, K), arginine (Arg, R) and tyrosine (Tyr, Y);
heavy chain CDR2 (V_{H}-CDR2) with the amino acid sequence WINPDSGGTNYAQKFQG (SEQ ID NO: 34); and
heavy chain CDR3 (V_{H}-CDR3) with the amino acid sequence DQPLGYCTNGVCSYFDY (SEQ ID NO: 35).

In some embodiments, X₁ in V_{H}-CDR1 is lysine (Lys, K). In some embodiments, X₁ in V_{H}-CDR1 is arginine (Arg, R). In some embodiments, X₁ in V_{H}-CDR1 is tyrosine (Tyr, Y).

In some embodiments, the conditionally-active antibodies or antigen-binding fragments thereof comprise the following CDR sequences (as defined by Chothia numbering):
light chain CDR1 (V_{L}-CDR1) with the amino acid sequence RASQGIYSWLA (SEQ ID NO: 31);
light chain CDR2 (V_{L}-CDR2) with the amino acid sequence TASTLQS (SEQ ID NO: 32);
and
light chain CDR3 (V_{L}-CDR3) with the amino acid sequence QQANIFPLT (SEQ ID NO: 10); and
heavy chain CDR1 (V_{H}-CDR1) with the amino acid sequence GYTFTGX₁ (SEQ ID NO: 36), wherein X₁ is selected from the group consisting of lysine (Lys, K), arginine (Arg, R) and tyrosine (Tyr, Y);
heavy chain CDR2 (V_{H}-CDR2) with the amino acid sequence NPDSGG (SEQ ID NO: 37); and
heavy chain CDR3 (V_{H}-CDR3) with the amino acid sequence DQPLGYCTNGVCSYFDY (SEQ ID NO: 35).

In some embodiments, X₁ in V_{H}-CDR1 is lysine (Lys, K). In some embodiments, X₁ in V_{H}-CDR1 is arginine (Arg, R). In some embodiments, X₁ in V_{H}-CDR1 is tyrosine (Tyr, Y).

In some embodiments, the conditionally-active antibodies or antigen-binding fragments thereof comprise:
a light chain variable region sharing at least 70%, 75%, 80%, 85%, 90%, 95%, 96%, 97%, 98%, or 99% of sequence identity over the non-CDR regions of SEQ ID NO: 1; and
a heavy chain variable region sharing at least 70%, 75%, 80%, 85%, 90%, 95%, 96%, 97%, 98%, or 99% of sequence identity over the non-CDR regions of SEQ ID NO: 5, 6, 7, 8, or 2.

In some embodiments, the conditionally-active antibodies or antigen-binding fragments thereof comprise:
a light chain variable region with SEQ ID NO: 1; and
a heavy chain variable region with SEQ ID NO: 5, 6, 7, 8, or 2.

In some embodiments, the conditionally-active antibodies or antigen-binding fragments thereof comprise:
a light chain variable region sharing at least 70%, 75%, 80%, 85%, 90%, 95%, 96%, 97%, 98%, or 99% of sequence identity over the non-CDR regions of SEQ ID NO: 1; and
a heavy chain variable region sharing at least 70%, 75%, 80%, 85%, 90%, 95%, 96%, 97%, 98%, or 99% of sequence identity over the non-CDR regions of SEQ ID NO: 7 or 8 or 2.

In some embodiments, the conditionally-active antibodies or antigen-binding fragments thereof comprise:
a light chain variable region with SEQ ID NO: 1; and
a heavy chain variable region with SEQ ID NO: 7 or 8 or 2.

In some embodiments, the conditionally-active antibodies or antigen-binding fragments thereof further comprise a substitution in the third heavy chain framework region (V_{H}-FR3), at position S77 of SEQ ID NO: 7 or 8 or 2. In some embodiments, this substitution is selected from the group consisting of S77A and S77P.

In some embodiments, the conditionally-active antibodies or antigen-binding fragments thereof comprise:
a light chain variable region sharing at least 70%, 75%, 80%, 85%, 90%, 95%, 96%, 97%, 98%, or 99% of sequence identity over the non-CDR regions of SEQ ID NO: 1; and
a heavy chain variable region sharing at least 70%, 75%, 80%, 85%, 90%, 95%, 96%, 97%, 98%, or 99% of sequence identity over the non-CDR regions of SEQ ID NO: 5 or 6, wherein position 77 of SEQ ID NO: 5 or 6 retains an alanine (Ala, A) or a proline (Pro, P) residue.

In some embodiments, the conditionally-active antibodies or antigen-binding fragments thereof comprise:
a light chain variable region with SEQ ID NO: 1; and
a heavy chain variable region with SEQ ID NO: 5 or 6.

In some embodiments, the at least one masking moiety is a peptide comprising from about 10 to about 50 amino acid residues, from about 10 to about 40 amino acid residues, from about 10 to about 30 amino acid residues, from about 15 to about 25 amino acid residues.

In some embodiments, the at least one masking moiety, when coupled to the antibodies or antigen-binding fragments thereof, reduces or inhibits binding of the antibodies or antigen-binding fragments thereof to its target antigen. The at least one masking moiety may act by partially or totally shielding the paratope of the antibodies or antigen-binding fragments thereof, by creating steric hindrance between the antibodies or antigen-binding fragments thereof and their target, and the like.

In some embodiments, the at least one masking moiety is coupled to the N-terminus of the light chain variable region of the conditionally-active antibodies or antigen-binding fragments thereof.

In some embodiments, the at least one masking moiety comprises or consists of an amino acid sequence with SEQ ID NO: 22 or 23 or an amino acid sequence sharing at least 70%, 75%, 80%, 85%, 90%, 95%, 96%, 97%, 98%, or 99% of sequence identity with SEQ ID NO: 22 or 23.

In some embodiments, the conditionally-active antibodies or antigen-binding fragments thereof further comprise at least one cleavable linker between the at least one masking moiety and the antibody or antigen-binding fragment thereof.

In some embodiments, the at least one cleavable linker is cleavable by at least one tumor-specific protease. The at least one tumor-specific protease may be selected from the group consisting of matrix metalloproteinase-9 (MMP-9), urokinase-type plasminogen activator (uPa), matrix metalloproteinase-2 (MMP-2), matriptase, legumain, kallikrein-related peptidase-3, human neutrophil elastase, proteinase 3 (Pr3), cathepsin B, and cathepsin K.

In some embodiments, the at least one tumor-specific protease is MMP-9 or uPa, or a combination thereof.

In some embodiments, the at least one cleavable linker comprises an amino acid sequence with SEQ ID NO: 38 and/or 39.

In some embodiments, the at least one cleavable linker comprises or consists of an amino acid sequence with SEQ ID NO: 24 or 25; or an amino acid sequence sharing at least 70%, 75%, 80%, 85%, 90%, 95%, 96%, 97%, 98%, or 99% of sequence identity with SEQ ID NO: 24 or 25 and retaining its ability to be cleaved by least one tumor-specific protease.

Further examples of linkers which are cleavable by at least one tumor-specific protease are known in the art. The skilled artisan will readily be able to select other suitable amino acid sequences cleavable by tumor-specific proteases.

In some embodiments, the conditionally-active antibodies or antigen-binding fragments thereof further comprise:
a light chain variable region sharing at least 70%, 75%, 80%, 85%, 90%, 95%, 96%, 97%, 98%, or 99% of sequence identity over the non-CDR regions of SEQ ID NO: 26 or 27 or 28 or 29; and
a heavy chain variable region sharing at least 70%, 75%, 80%, 85%, 90%, 95%, 96%, 97%, 98%, or 99% of sequence identity over the non-CDR regions of SEQ ID NO: 5 or 6 or 7 or 8 or 2.

In some embodiments, the conditionally-active antibodies or antigen-binding fragments thereof comprise:
a light chain variable region with SEQ ID NO: 26 or 27 or 28 or 29; and
a heavy chain variable region with SEQ ID NO: 5 or 6 or 7 or 8 or 2.

In some embodiments, the conditionally-active antibodies or antigen-binding fragments thereof comprise:
a light chain variable region with SEQ ID NO: 26; and
a heavy chain variable region with SEQ ID NO: 5 or 6 or 7 or 8 or 2.

In some embodiments, the conditionally-active antibodies or antigen-binding fragments thereof comprise:
a light chain variable region with SEQ ID NO: 27; and
a heavy chain variable region with SEQ ID NO: 5 or 6 or 7 or 8 or 2.

In some embodiments, the conditionally-active antibodies or antigen-binding fragments thereof comprise:
a light chain variable region with SEQ ID NO: 28; and
a heavy chain variable region with SEQ ID NO: 5 or 6 or 7 or 8 or 2.

In some embodiments, the conditionally-active antibodies or antigen-binding fragments thereof comprise:
a light chain variable region with SEQ ID NO: 29; and
a heavy chain variable region with SEQ ID NO: 5 or 6 or 7 or 8 or 2.

In some embodiments, the conditionally-active antibodies or antigen-binding fragments thereof described herein are capable of binding to CD40, preferably to human CD40, upon cleavage of the at least one cleavable linker and release of the at least one masking moiety.

In a third aspect, conditionally-active antibodies or antigen-binding fragments thereof capable of specifically binding to CD40 are provided, wherein the conditionally-active antibodies or antigen-binding fragments thereof are coupled to at least one masking moiety.

In some embodiments, the at least one masking moiety, when coupled to the antibodies or antigen-binding fragments thereof, reduces or inhibits binding of the antibodies or antigen-binding fragments thereof to CD40. The at least one masking moiety may act by partially or totally shielding the paratope of the antibodies or antigen-binding fragments thereof, by creating steric hindrance between the antibodies or antigen-binding fragments thereof and their target, and the like.

In some embodiments, the conditionally-active antibodies or antigen-binding fragments thereof further comprise at least one cleavable linker between the at least one masking moiety and the antibody or antigen-binding fragment thereof.

In some embodiments, the at least one cleavable linker is cleavable by at least one tumor-specific protease.

The at least one tumor-specific protease may be selected from the group consisting of matrix metalloproteinase-9 (MMP-9), urokinase-type plasminogen activator (uPa), matrix metalloproteinase-2 (MMP-2), matriptase, legumain, kallikrein-related peptidase-3, human neutrophil elastase, proteinase 3 (Pr3), cathepsin B, and cathepsin K.

In some embodiments, the at least one tumor-specific protease is MMP-9 or uPa, or a combination thereof.

In some embodiments, the at least one cleavable linker comprises an amino acid sequence with SEQ ID NO: 38 and/or 39.

In some embodiments, the at least one cleavable linker comprises or consists of an amino acid sequence with SEQ ID NO: 24 or 25 or an amino acid sequence sharing at least 70%, 75%, 80%, 85%, 90%, 95%, 96%, 97%, 98%, or 99% of sequence identity with SEQ ID NO: 24 or 25 and retaining its ability to be cleaved by least one tumor-specific protease.

Further examples of linkers which are cleavable by at least one tumor-specific protease are known in the art. The skilled artisan will readily be able to select other suitable amino acid sequences cleavable by tumor-specific proteases.

In some embodiments, the conditionally-active antibodies or antigen-binding fragments thereof described herein are capable of binding to CD40, preferably to human CD40, upon cleavage of the at least one cleavable linker and release of the at least one masking moiety.

In a fourth aspect, conditionally-active antibodies or antigen-binding fragments thereof capable of specifically binding to CD40 are provided, wherein the conditionally-active antibodies or antigen-binding fragments thereof are coupled to at least one masking moiety comprising or consisting of an amino acid sequence with SEQ ID NO: 22 or 23 or an amino acid sequence sharing at least 70%, 75%, 80%, 85%, 90%, 95%, 96%, 97%, 98%, or 99% of sequence identity with SEQ ID NO: 22 or 23.

In some embodiments, the at least one masking moiety, when coupled to the antibodies or antigen-binding fragments thereof, reduces or inhibits binding of the antibodies or antigen-binding fragments thereof to CD40. The at least one masking moiety may act by partially or totally shielding the paratope of the antibodies or antigen-binding fragments thereof, by creating steric hindrance between the antibodies or antigen-binding fragments thereof and their target, and the like.

In some embodiments, the conditionally-active antibodies or antigen-binding fragments thereof further comprise at least one cleavable linker between the at least one masking moiety and the antibody or antigen-binding fragment thereof.

In some embodiments, the at least one cleavable linker is cleavable by at least one tumor-specific protease.

The at least one tumor-specific protease may be selected from the group consisting of matrix metalloproteinase-9 (MMP-9), urokinase-type plasminogen activator (uPa), matrix metalloproteinase-2 (MMP-2), matriptase, legumain, kallikrein-related peptidase-3, human neutrophil elastase, proteinase 3 (Pr3), cathepsin B, and cathepsin K.

In some embodiments, the at least one tumor-specific protease is MMP-9 or uPa, or a combination thereof.

In some embodiments, the at least one cleavable linker comprises an amino acid sequence with SEQ ID NO: 38 and/or 39.

In some embodiments, the at least one cleavable linker comprises or consists of an amino acid sequence with SEQ ID NO: 24 or 25 or an amino acid sequence sharing at least 70%, 75%, 80%, 85%, 90%, 95%, 96%, 97%, 98%, or 99% of sequence identity with SEQ ID NO: 24 or 25 and retaining its ability to be cleaved by least one tumor-specific protease.

Further examples of linkers which are cleavable by at least one tumor-specific protease are known in the art. The skilled artisan will readily be able to select other suitable amino acid sequences cleavable by tumor-specific proteases.

In some embodiments, the conditionally-active antibodies or antigen-binding fragments thereof described herein are capable of binding to CD40, preferably to human CD40, upon cleavage of the at least one cleavable linker and release of the at least one masking moiety.

In some embodiments applicable to all of the above aspects, the antibodies or antigen-binding fragments thereof described herein, and the conditionally-active antibodies or antigen-binding fragments thereof described herein, have pure agonist activity. In some embodiments, pure agonist activity means that the antibody or antigen-binding fragment thereof or the conditionally-active antibody or antigen-binding fragment thereof is capable of clustering CD40 on the surface of a cell and of activating the CD40 signaling pathway in an FcγR-independent manner or independently of any other type of target-mediated crosslinking. In some embodiments, pure agonist activity means means that the antibody or antigen-binding fragment thereof or the conditionally-active antibody or antigen-binding fragment thereof is capable of activating the CD40 signaling pathway (i) in soluble conditions, and/or (ii) in the absence of a cross-linking reagent, and/or (iii) in a FcγR-independent manner, and/or (iv) in the absence of target-mediated crosslinking of CD40.

### Antigen-Binding Fragments of Antibodies

Unless specifically indicated otherwise, the term “antibody”, as used herein, shall be understood to encompass antibody molecules comprising two immunoglobulin heavy chains and two immunoglobulin light chains (i.e., “full antibody molecules”) as well as antigen-binding fragments thereof. An antibody fragment may include a Fab fragment, a F(ab′)2 fragment, a Fv fragment, a dAb fragment, a fragment containing a CDR, or an isolated CDR. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment”, as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}—CHI-C_{H}2; (v) V_{H}-C_{H}1-C_{H}2-C_{H}3; (vi) V_{H}-C_{H}2-C_{H}3; (vii) V_{H}-CL; (viii) VL-CHI; (ix) VL-C_{H}2; (x) VL-C_{H}3; (xi) VL-CHI-C_{H}2; (xii) VL-C_{H}1-C_{H}2-C_{H}3; (xiii) VL-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multi-specific antibody format, including the exemplary bi-specific antibody formats disclosed herein, may be adapted for use in the context of an antigen-binding fragment of an antibody of the present disclosure using routine techniques available in the art.

### Preparation of Human Antibodies

Methods for generating human antibodies in transgenic mice are known in the art. Any such known methods can be used in the context of the present disclosure to make human antibodies that specifically bind to CD40.

An immunogen comprising any one of the following can be used to generate antibodies to CD40. In certain embodiments, the antibodies of the disclosure are obtained from mice immunized with a full length, native CD40, or with a recombinant CD40 peptide. Alternatively, CD40 or a fragment thereof may be produced using standard biochemical techniques and modified and used as immunogen. In certain embodiments, the immunogen may be a peptide from the N terminal or C terminal end of CD40.

In some embodiments, the immunogen may be a recombinant CD40 peptide expressed in E. coli or in any other eukaryotic or mammalian cells such as Chinese hamster ovary (CHO) cells.

In certain embodiments, antibodies that bind specifically to CD40 may be prepared using fragments of the above-noted regions, or peptides that extend beyond the designated regions by about 5 to about 20 amino acid residues from either, or both, the N or C terminal ends of the regions described herein. In certain embodiments, any combination of the above-noted regions or fragments thereof may be used in the preparation of CD40-specific antibodies.

Using VELOCIMMUNE® technology (see, for example, U.S. Pat. No. 6,596,541, Regeneron Pharmaceuticals, VELOCIMMUNE®) or any other known method for generating monoclonal antibodies, high affinity chimeric antibodies to CD40 can be initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

### Bioequivalents

The anti-CD40 antibodies of the present disclosure encompass proteins having amino acid sequences that vary from those of the described antibodies, but that retain the ability to bind CD40. Such variant antibodies and antigen-binding fragments thereof comprise one or more additions, deletions, or substitutions of amino acids when compared to parent sequence, but exhibit biological activity that is essentially equivalent to that of the described antibodies. Likewise, the antibody-encoding DNA sequences of the present disclosure encompass sequences that comprise one or more additions, deletions, or substitutions of nucleotides when compared to the disclosed sequence, but that encode an antibody or antibody fragment that is essentially bioequivalent to an antibody or antibody fragment of the disclosure.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single dose or multiple doses. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, or potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and/or in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the antibodies of the disclosure may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include antibody variants comprising amino acid changes, which modify the glycosylation characteristics of the antibodies, e.g., mutations that eliminate or remove glycosylation.

### Anti-CD40 Antibodies Comprising Fc Variants

According to certain embodiments of the present disclosure, anti-CD40 antibodies are provided comprising an Fc domain comprising one or more mutations which enhance or diminish antibody binding to the FcRn receptor, e.g., at acidic pH as compared to neutral pH. For example, the present disclosure includes anti-CD40 antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 234 (e.g., A), 235 (e.g., A), 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., A, W, H, F or Y [N434A, N434W, N434H, N434F or N434Y]); or a modification at position 250 and/or 428; or a modification at position 307 or 308 (e.g., 308F, V308F), and 434. In one embodiment, the modification comprises a 234A (e.g., L234A) and 235A (e.g., L235A) modification, 428L (e.g., M428L) and 434S (e.g., N434S) modification; a 428L, 2591 (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P). In yet another embodiment, the modification comprises a 265A (e.g., D265A) and/or a 297A (e.g., N297A) modification.

For example, the present disclosure includes anti-CD40 antibodies comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); 2571 and 3111 (e.g., P257I and Q3111); 2571 and 434H (e.g., P257I and N434H); 376V and 434H (e.g., D376V and N434H); 307A, 380A and 434A (e.g., T307A, E380A and N434A); 433K and 434F (e.g., H433K and N434F); and 234A and 235A (e.g., L234A and L235A). In one embodiment, the present disclosure includes anti-CD40 antibodies comprising an Fc domain comprising a S108P mutation in the hinge region of IgG4 to promote dimer stabilization. All possible combinations of the foregoing Fc domain mutations, and other mutations within the antibody variable domains disclosed herein, are contemplated within the scope of the present disclosure.

The present disclosure also includes anti-CD40 antibodies comprising a chimeric heavy chain constant (C_{H}) region, wherein the chimeric C_{H} region comprises segments derived from the C_{H} regions of more than one immunoglobulin isotype. For example, the antibodies of the disclosure may comprise a chimeric C_{H} region comprising part or all of a C_{H}2 domain derived from a human IgG1, human IgG2 or human IgG4 molecule, combined with part or all of a C_{H}3 domain derived from a human IgG1, human IgG2 or human IgG4 molecule. According to certain embodiments, the antibodies of the disclosure comprise a chimeric C_{H} region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antibody comprising a chimeric C_{H} region as described herein may, in certain embodiments, exhibit modified Fe effector functions without adversely affecting the therapeutic or pharmacokinetic properties of the antibody. See, e.g., U.S. Pat. No. 9,359,437, the disclosure of which is hereby incorporated by reference in its entirety.

### Biological Characteristics of the Antibodies

In general, the antibodies of the present disclosure function by binding to CD40. The present disclosure includes anti-CD40 antibodies and antigen-binding fragments thereof that bind CD40 molecules with high affinity. For example, the present disclosure includes antibodies and antigen-binding fragments of antibodies that CD40 (e.g., at 25° C. or at 37° C.) with a K_{D} of less than about 50 nM as measured by surface plasmon resonance. In certain embodiments, the antibodies or antigen-binding fragments thereof bind CD40 with a K_{D} of less than about 40 nM, less than about 30 nM, less than about 20 nM, less than about 10 nM less than about 5 nM, less than about 2 nM or less than about 1 nM, as measured by surface plasmon resonance.

The present disclosure also includes antibodies and antigen-binding fragments thereof that bind CD40 with a dissociative half-life (t½) of greater than about 1.1 minutes as measured by surface plasmon resonance at 25° C. or 37° C. In certain embodiments, the antibodies or antigen-binding fragments of the present disclosure bind CD40 with a t½ of greater than about 5 minutes, greater than about 10 minutes, greater than about 30 minutes, greater than about 50 minutes, greater than about 60 minutes, greater than about 70 minutes, greater than about 80 minutes, greater than about 90 minutes, greater than about 100 minutes, greater than about 200 minutes, greater than about 300 minutes, greater than about 400 minutes, greater than about 500 minutes, greater than about 600 minutes, greater than about 700 minutes, greater than about 800 minutes, greater than about 900 minutes, greater than about 1000 minutes, or greater than about 1200 minutes, as measured by surface plasmon resonance at 25° C. or 37° C.

### Species Selectivity and Species Cross-Reactivity

According to certain embodiments of the disclosure, the anti-CD40 antibodies bind to human CD40 but not to CD40 from other species. Alternatively, the anti-CD40 antibodies of the disclosure, in certain embodiments, bind to human CD40 and to CD40 from one or more non-human species. For example, the anti-CD40 antibodies of the disclosure may bind to human CD40 and may bind or not bind, as the case may be, to one or more of mouse, rat, guinea pig, hamster, gerbil, pig, cat, dog, rabbit, goat, sheep, cow, horse, camel, cynomolgus, marmoset, rhesus or chimpanzee CD40. In certain embodiments, the anti-CD40 antibodies of the disclosure may bind to human and cynomolgus CD40 with the same affinities or with different affinities, but do not bind to rat and mouse CD40.

### Immunoconjugates

The disclosure encompasses anti-CD40 antibodies conjugated to a therapeutic moiety (“immunoconjugate”), such as a cytotoxin or a chemotherapeutic agent to treat cancer. As used herein, the term “immunoconjugate” refers to an antibody which is chemically or biologically linked to a cytotoxin, a radioactive agent, a cytokine, an interferon, a target or reporter moiety, an enzyme, a toxin, a peptide or protein or a therapeutic agent. The antibody may be linked to the cytotoxin, radioactive agent, cytokine, interferon, target or reporter moiety, enzyme, toxin, peptide or therapeutic agent at any location along the molecule so long as it is able to bind its target.

Examples of immunoconjugates include antibody drug conjugates and antibody-toxin fusion proteins. In one embodiment, the agent may be a second different antibody to CD40. In certain embodiments, the antibody may be conjugated to an agent specific for a tumor cell or a virally infected cell. The type of therapeutic moiety that may be conjugated to the anti-CD40 antibodies and will take into account the condition to be treated and the desired therapeutic effect to be achieved. Examples of suitable agents for forming immunoconjugates are known in the art; see for example, WO 05/103081.

### Therapeutic Administration and Formulations

The disclosure provides therapeutic compositions comprising the anti-CD40 antibodies of the present disclosure. Therapeutic compositions in accordance with the disclosure will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al., PDA J Pharm Sci Technol. 1998 Sep-Oct;52(5):238-311.

The dose of antibody may vary depending upon the age and the size of a subject to be administered, target disease, conditions, route of administration, and the like. When an antibody of the present disclosure is used for treating a disease or disorder in an adult patient, or for preventing such a disease, it is advantageous to administer the antibody of the present disclosure normally at a single dose of about 0.1 to about 60 mg/kg body weight, about 5 to about 60, about 10 to about 50, or about 20 to about 50 mg/kg body weight. Depending on the severity of the condition, the frequency and the duration of the treatment can be adjusted. In certain embodiments, the antibody or antigen-binding fragment thereof of the disclosure can be administered as an initial dose of at least about 0.1 mg to about 800 mg, about 1 to about 500 mg, about 5 to about 300 mg, or about 10 to about 200 mg, to about 100 mg, or to about 50 mg. In certain embodiments, the initial dose may be followed by administration of a second or a plurality of subsequent doses of the antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., J Biol Chem. 1987 Apr. 5; 262(10):4429-32). Methods of introduction include, but are not limited to, intradermal, transdermal, intramuscular, intraperitoneal, intravenous, subcutaneous, intranasal, intratumoral, epidural and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The pharmaceutical composition can be also delivered in a vesicle, in particular a liposome (see, for example, Langer. Science. 1990 Sep. 28; 249(4976):1527-33).

The use of nanoparticles to deliver the antibodies of the present disclosure is also contemplated herein. Antibody-conjugated nanoparticles may be used both for therapeutic and diagnostic applications. Antibody-conjugated nanoparticles and methods of preparation and use are described in detail by Arruebo, M., et al. 2009 (“Antibody-conjugated nanoparticles for biomedical applications” in J. Nanomat. Volume 2009, Article ID 439389, 24 pages, doi: 10.1155/2009/439389), incorporated herein by reference. Nanoparticles may be developed and conjugated to antibodies contained in pharmaceutical compositions to target tumor cells or autoimmune tissue cells or virally infected cells. Nanoparticles for drug delivery have also been described in, for example, U.S. Pat. No. 8,257,740, or U.S. Pat. No. 8,246,995, each incorporated herein in its entirety.

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracutaneous, intratumoral, intracranial, intraperitoneal and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is optionally filled in an appropriate ampule.

A pharmaceutical composition of the present disclosure can be delivered subcutaneously, intravenously or intratumorally with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present disclosure. Examples include, but certainly are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (Sanofi-Aventis, Frankfurt, Germany), and the like. Examples of disposable pen delivery devices having applications in subcutaneous delivery of a pharmaceutical composition of the present disclosure include, but certainly are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L. P.) and the HUMIRA™ Pen (Abbott Labs, Abbott Park, Ill.), and the like.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the antibody contained is generally about 5 to about 500 mg per dosage form in a unit dose; especially in the form of injection, the antibody is contained in about 5 to about 100 mg and in about 10 to about 250 mg for the other dosage forms.

### Therapeutic Uses of the Antibodies

The antibodies of the disclosure are useful, inter alia, for the treatment, prevention and/or amelioration of any disease or disorder associated with or mediated by CD40 expression, signaling, or activity, or treatable by mimicking the action of a CD40 ligand (e.g., CD40L) or otherwise activating CD40 activity and/or signaling pathway. For example, the present disclosure provides methods for treating cancer (tumor growth inhibition) and/or chronic viral infections by administering an anti-CD40 antibody (or pharmaceutical composition comprising anti-CD40 antibody) as described herein to a patient in need of such treatment. The antibodies of the present disclosure are useful for the treatment, prevention, and/or amelioration of disease or disorder or condition such as cancer or viral infections, and/or for ameliorating at least one symptom associated with such disease, disorder or condition. In the context of the methods of treatment described herein, the anti-CD40 antibody may be administered as a monotherapy (i.e., as the only therapeutic agent) or in combination with one or more additional therapeutic agents (examples of which are described elsewhere herein).

In some embodiments of the disclosure, the antibodies described herein are useful for treating subjects suffering from primary or recurrent cancer, including, but not limited to, bladder cancer, bone cancer, brain cancer, breast cancer, cervical cancer, colon cancer, esophageal cancer, gastric cancer, head-and-neck cancer, leukemia, liver cancer, lung cancer, lymphoma, melanoma, mesothelioma, multiple myeloma, myelodysplastic syndrome, ovarian cancer, pancreatic cancer, prostate cancer, rectal cancer, renal/kidney cancer, sarcoma, skin cancer, testicular cancer, thyroid cancer, and uterine cancer.

The antibodies may be used to treat early stage or late-stage symptoms of cancer. In one embodiment, an antibody or fragment thereof of the disclosure may be used to treat metastatic cancer. The antibodies are useful in reducing or inhibiting or shrinking tumor growth of both solid tumors and blood cancers. In certain embodiments, treatment with an antibody or antigen-binding fragment thereof of the disclosure leads to more than 50% regression, more than 60% regression, more than 70% regression, more than 80% regression or more than 90% regression of a tumor in a subject. In certain embodiments, the antibodies may be used to prevent relapse of a tumor. In certain embodiments, the antibodies are useful in extending overall survival in a subject with cancer. In some embodiments, the antibodies are useful in reducing toxicity due to chemotherapy or radiotherapy while maintaining long-term survival in a patient suffering from cancer.

In certain embodiments, the antibodies of the disclosure are useful to treat subjects suffering from a chronic viral infection. In some embodiments, the antibodies of the disclosure are useful in decreasing viral titers in the host and/or rescuing exhausted T-cells. In certain embodiments, an antibody or fragment thereof of the disclosure may be used to treat chronic viral infection by lymphocytic choriomeningitis virus (LCMV). In some embodiments, an antibody or antigen-binding fragment thereof the disclosure may be administered at a therapeutic dose to a patient with an infection by human immunodeficiency virus (HIV) or human papilloma virus (HPV) or hepatitis B/C virus (HBV/HCV). In a related embodiment, an antibody or antigen-binding fragment thereof of the disclosure may be used to treat an infection by simian immunodeficiency virus (SIV) in a simian subject such as cynomolgus.

In certain embodiments, an antibody of the present disclosure may be administered in a therapeutically effective amount to a subject suffering from a cancer or a viral infection.

One or more antibodies of the present disclosure may be administered to relieve or prevent or decrease the severity of one or more of the symptoms or conditions of the disease or disorder.

It is also contemplated herein to use one or more antibodies of the present disclosure prophylactically to patients at risk for developing a disease or disorder such as cancer and/or chronic viral infections.

In a further embodiment of the disclosure the present antibodies are used for the preparation of a pharmaceutical composition for treating patients suffering from cancer and/or viral infection. In another embodiment of the disclosure, the present antibodies are used as adjunct therapy with any other agent or any other therapy known to those skilled in the art useful for treating cancer and/or viral infection.

### Combination Therapies and Formulations

Combination therapies may include an anti-CD40 antibody of the disclosure and any additional therapeutic agent that may be advantageously combined with an antibody of the disclosure.

The antibodies of the present disclosure may be combined synergistically with one or more anti-cancer drugs or therapy used to treat cancer, including, for example, bladder cancer, bone cancer, brain cancer, breast cancer, cervical cancer, colon cancer, esophageal cancer, gastric cancer, head-and-neck cancer, leukemia, liver cancer, lung cancer, lymphoma, melanoma, mesothelioma, multiple myeloma, myelodysplastic syndrome, ovarian cancer, pancreatic cancer, prostate cancer, rectal cancer, renal/kidney cancer, sarcoma, skin cancer, testicular cancer, thyroid cancer, and uterine cancer. It is contemplated herein to use anti-CD40 antibodies of the disclosure in combination with immunostimulatory and/or immunosupportive therapies to inhibit tumor growth, and/or enhance survival of cancer patients. The immunostimulatory therapies include direct immunostimulatory therapies to augment immune cell activity by either “releasing the brake” on suppressed immune cells or “stepping on the gas” to activate an immune response. Examples include targeting other checkpoint receptors, adoptive cell therapy, vaccination and adjuvants. The immunosupportive modalities may increase antigenicity of the tumor by promoting immunogenic cell death, inflammation or have other indirect effects that promote an anti-tumor immune response. Examples include radiation, chemotherapy, anti-angiogenic agents, and surgery.

In various embodiments, one or more antibodies of the present disclosure may be used in combination with an antibody to CD40L; a second antibody to CD40; a LAG-3 inhibitor; a CTLA-4 inhibitor (e.g., ipilimumab); a TIM-3 inhibitor; a BTLA inhibitor; a TIGIT inhibitor; a CD47 inhibitor; an antagonist of a T-cell co-inhibitor or ligand (e.g., an antibody to PD-1, PD-L1, PD-L2, CEACAM, VISTA, LAIR-1, 2B4, B7-H3, B7-H4, KIR, A2aR, GAL9, or TGFR); an agonist of a T-cell co-stimulator (e.g., an antibody or a ligand to 4-1BB, CD28, ICOS, OX40, CD27, B7, CD226, CRTAM, GITR, HVEM, BAFFR, BAFF, Light); adenosine; an indoleamine-2,3-dioxygenase (IDO) inhibitor; a vascular endothelial growth factor (VEGF) antagonist (e.g., a “VEGF-Trap” such as aflibercept or other VEGF-inhibiting fusion protein as set forth in U.S. Pat. No. 7,087,411, or an anti-VEGF antibody or antigen binding fragment thereof [e.g., bevacizumab, or ranibizumab] or a small molecule kinase inhibitor of VEGF receptor [e.g., sunitinib, sorafenib, or pazopanib]); an Ang2 inhibitor (e.g., nesvacumab); a transforming growth factor beta (TGFO) inhibitor; an epidermal growth factor receptor (EGFR) inhibitor (e.g., erlotinib, cetuximab); an agonist to a co-stimulatory receptor (e.g., an agonist to glucocorticoid-induced TNFR-related protein); an antibody to a tumor-specific antigen (e.g., CA9, CA125, melanoma-associated antigen 3 [MAGE3], carcinoembryonic antigen [CEA], vimentin, tumor-M2-PK, prostate-specific antigen [PSA], mucin-1, MART-1, and CA19⁻⁹); a vaccine (e.g., Bacillus Calmette-Guerin, a cancer vaccine); an adjuvant to increase antigen presentation (e.g., granulocyte-macrophage colony-stimulating factor); a bispecific antibody (e.g., CD3×CD20 bispecific antibody, PSMAxCD3 bispecific antibody); a cytotoxin; a chemotherapeutic agent (e.g., dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, and vincristine); cyclophosphamide; radiotherapy; an IL-6R inhibitor (e.g., sarilumab); an IL-4R inhibitor (e.g., dupilumab); an IL-10 inhibitor; a cytokine such as IL-2, IL-7, IL-12, IL-21, and IL-15; an antibody-drug conjugate (ADC) (e.g., anti-CD19-DM4 ADC, and anti-DS6-DM4 ADC); an immunocytokine (e.g., an anti-FAPxIL-2v [e.g., RO6874281], anti-tenascin CxIL-2 [e.g., F16-IL2, a.k.a. teleukin], anti-GD2×IL-2 [e.g., hul4.18-IL2], anti-EDBxIL-2 [e.g., L19-IL2, a.k.a. darleukin], anti-EDB×TNF [e.g., L19-TNF, a.k.a. fibromun], anti-histone complexxIL-12 [e.g., NHS-IL12], anti-EDBxIL-12 [e.g., L19-IL12, a.k.a. dodekin], anti-CSPG4×IL-2, anti-EpCAM×IL-2, anti-CD20×IL-2, anti-PD-1×IL-2, and anti-TNFα×IL-2); an anti-inflammatory drug (e.g., corticosteroids, and non-steroidal anti-inflammatory drugs); a dietary supplement such as anti-oxidants; or any palliative care to treat cancer. In certain embodiments, the anti-CD40 antibodies of the present disclosure may be used in combination with 5i cancer vaccines (including, without limitation, dendritic cell vaccines, oncolytic viruses, tumor cell vaccines, etc.), or adoptive cell therapies, to augment the anti-tumor response. Examples of cancer vaccines that can be used in combination with anti-CD40 antibodies of the present disclosure include MAGE3 vaccine for melanoma and bladder cancer, MUC1 vaccine for breast cancer, EGFRv3 (e.g., Rindopepimut) for brain cancer (including glioblastoma multiforme), or ALVAC-CEA (for CEA+cancers).

In certain embodiments, the anti-CD40 antibodies of the disclosure may be administered in combination with radiation therapy in methods to generate long-term durable anti-tumor responses and/or enhance survival of patients with cancer. In some embodiments, the anti-CD40 antibodies of the disclosure may be administered prior to, concomitantly or after administering radiation therapy to a cancer patient. For example, radiation therapy may be administered in one or more doses to tumor lesions followed by administration of one or more doses of anti-CD40 antibodies of the disclosure. In some embodiments, radiation therapy may be administered locally to a tumor lesion to enhance the local immunogenicity of a patient's tumor (adjuvinating radiation) and/or to kill tumor cells (ablative radiation) followed by systemic administration of anti-CD40 antibody of the disclosure. For example, intracranial radiation may be administered to a patient with brain cancer (e.g., glioblastoma multiforme) in combination with systemic administration of an anti-CD40 antibody of the disclosure. In certain embodiments, the anti-CD40 antibodies of the disclosure may be administered in combination with radiation therapy and a chemotherapeutic agent (e.g., temozolomide) or a VEGF antagonist (e.g., aflibercept).

In certain embodiments, the anti-CD40 antibodies of the disclosure may be administered in combination with one or more anti-viral drugs to treat chronic viral infection caused by LCMV, HIV, HPV, HBV or HCV. Examples of anti-viral drugs include, but are not limited to, zidovudine, lamivudine, abacavir, ribavirin, lopinavir, efavirenz, cobicistat, tenofovir, rilpivirine and corticosteroids. In some embodiments, the anti-CD40 antibodies of the disclosure may be administered in combination with a LAG3 inhibitor, a CTLA-4 inhibitor, or any antagonist of another T-cell co-inhibitor to treat chronic viral infection.

The additional therapeutically active agent(s)/component(s) may be administered prior to, concurrent with, or after the administration of the anti-CD40 antibodies of the disclosure. For purposes of the present disclosure, such administration regimens are considered the administration of an anti-CD40 antibody “in combination with” a second therapeutically active component.

The additional therapeutically active component(s) may be administered to a subject prior to administration of an anti-CD40 antibody of the disclosure. For example, a first component may be deemed to be administered “prior to” a second component if the first component is administered 1 week before, 72 hours before, 60 hours before, 48 hours before, 36 hours before, 24 hours before, 12 hours before, 6 hours before, 5 hours before, 4 hours before, 3 hours before, 2 hours before, 1 hour before, 30 minutes before, 15 minutes before, 10 minutes before, 5 minutes before, or less than 1 minute before administration of the second component. In other embodiments, the additional therapeutically active component(s) may be administered to a subject after administration of an anti-CD40 antibody of the disclosure. For example, a first component may be deemed to be administered “after” a second component if the first component is administered 1 minute after, 5 minutes after, 10 minutes after, 15 minutes after, 30 minutes after, 1 hour after, 2 hours after, 3 hours after, 4 hours after, 5 hours after, 6 hours after, 12 hours after, 24 hours after, 36 hours after, 48 hours after, 60 hours after, 72 hours after administration of the second component. In yet other embodiments, the additional therapeutically active component(s) may be administered to a subject concurrent with administration of an anti-CD40 antibody of the disclosure. “Concurrent” administration, for purposes of the present disclosure, includes, e.g., administration of an anti-CD40 antibody and an additional therapeutically active component to a subject in a single dosage form (e.g., co-formulated), or in separate dosage forms administered to the subject within about 30 minutes or less of each other. If administered in separate dosage forms, each dosage form may be administered via the same route (e.g., both the anti-CD40 antibody and the additional therapeutically active component may be administered intravenously, intratumorally, subcutaneously, etc.); alternatively, each dosage form may be administered via a different route (e.g., the anti-CD40 antibody may be administered intravenously, and the additional therapeutically active component may be administered subcutaneously). In any event, administering the components in a single dosage from, in separate dosage forms by the same route, or in separate dosage forms by different routes are all considered “concurrent administration,” for purposes of the present disclosure. For purposes of the present disclosure, administration of an anti-CD40 antibody “prior to,” “concurrent with,” or “after” (as those terms are defined herein above) administration of an additional therapeutically active component is considered administration of an anti-CD40 antibody “in combination with” an additional therapeutically active component).

The present disclosure includes pharmaceutical compositions in which an anti-CD40 antibody of the disclosure is co-formulated with one or more of the additional therapeutically active component(s) as described elsewhere herein using a variety of dosage combinations.

### Administrative Regimens

According to certain embodiments of the present disclosure, multiple doses of an anti-CD40 antibody (or a pharmaceutical composition comprising a combination of an anti-CD40 antibody and any of the additional therapeutically active agents mentioned herein) may be administered to a subject over a defined time course. The methods according to this aspect of the disclosure comprise sequentially administering to a subject multiple doses of an anti-CD40 antibody of the disclosure. As used herein, “sequentially administering” means that each dose of anti-CD40 antibody is administered to the subject at a different point in time, e.g., on different days separated by a predetermined interval (e.g., hours, days, weeks or months). The present disclosure includes methods which comprise sequentially administering to the patient a single initial dose of an anti-CD40 antibody, followed by one or more secondary doses of the anti-CD40 antibody, and optionally followed by one or more tertiary doses of the anti-CD40 antibody. The anti-CD40 antibody may be administered at a dose between 0.1 mg/kg to 100 mg/kg.

The terms “initial dose”, “secondary doses”, and “tertiary doses”, refer to the temporal sequence of administration of the anti-CD40 antibody of the disclosure. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of anti-CD40 antibody, but generally may differ from one another in terms of frequency of administration. In certain embodiments, however, the amount of anti-CD40 antibody contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In certain exemplary embodiments of the present disclosure, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of anti-CD40 antibody which is administered to a patient prior to the administration of the very next dose in the sequence with no intervening doses.

The methods according to this aspect of the disclosure may comprise administering to a patient any number of secondary and/or tertiary doses of an anti-CD40 antibody. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks or 1 to 2 months after the immediately preceding dose. Similarly, in embodiments involving multiple tertiary doses, each tertiary dose may be administered at the same frequency as the other tertiary doses. For example, each tertiary dose may be administered to the patient 2 to 12 weeks after the immediately preceding dose. In certain embodiments of the disclosure, the frequency at which the secondary and/or tertiary doses are administered to a patient can vary over the course of the treatment regimen. The frequency of administration may also be adjusted during the course of treatment by a physician depending on the needs of the individual patient following clinical examination.

The present disclosure includes administration regimens in which 2 to 6 loading doses are administered to a patient at a first frequency (e.g., once a week, once every two weeks, once every three weeks, once a month, once every two months, etc.), followed by administration of two or more maintenance doses to the patient on a less frequent basis. For example, according to this aspect of the disclosure, if the loading doses are administered at a frequency of, e.g., once a month (e.g., two, three, four, or more loading doses administered once a month), then the maintenance doses may be administered to the patient once every five weeks, once every six weeks, once every seven weeks, once every eight weeks, once every ten weeks, once every twelve weeks, etc.).

### Diagnostic Uses of the Antibodies

An anti-CD40 antibody of the disclosure may be used to detect and/or measure CD40 in a sample, e.g., for diagnostic purposes. Some embodiments contemplate the use of one or more antibodies of the present disclosure in assays to detect a disease or disorder such as cancer, autoimmune disease, or chronic viral infection. Exemplary diagnostic assays for CD40 may comprise, e.g., contacting a sample, obtained from a patient, with an anti-CD40 antibody of the disclosure, wherein the anti-CD40 antibody is labeled with a detectable label or reporter molecule or used as a capture ligand to selectively isolate CD40 from patient samples. Alternatively, an unlabeled anti-CD40 antibody can be used in diagnostic applications in combination with a secondary antibody which is itself detectably labeled. The detectable label or reporter molecule can be a radioisotope, such as ³H, ¹⁴C, ³²P, ³S, or ¹²⁵I; a fluorescent or chemiluminescent moiety such as fluorescein isothiocyanate, or rhodamine; or an enzyme such as alkaline phosphatase, P-galactosidase, horseradish peroxidase, or luciferase. Specific exemplary assays that can be used to detect or measure CD40 in a sample include enzyme-linked immunosorbent assay (ELISA), radioimmunoassay (RIA), and fluorescence-activated cell sorting (FACS).

Samples that can be used in CD40 diagnostic assays according to the present disclosure include any tissue or fluid sample obtainable from a patient, which contains detectable quantities of CD40 protein, or fragments thereof, under normal or pathological conditions. Generally, levels of CD40 in a particular sample obtained from a healthy patient (e.g., a patient not afflicted with cancer, an autoimmune disease, or a chronic viral infection) will be measured to initially establish a baseline, or standard, level of CD40. This baseline level of CD40 can then be compared against the levels of CD40 measured in samples obtained from individuals suspected of having a cancer-related condition, an autoimmune disease, or a chronic viral infection; or symptoms associated with such condition.

The antibodies specific for CD40 may contain no additional labels or moieties, or they may contain an N-terminal or C-terminal label or moiety. In one embodiment, the label or moiety is biotin. In a binding assay, the location of a label (if any) may determine the orientation of the peptide relative to the surface upon which the peptide is bound. For example, if a surface is coated with avidin, a peptide containing an N-terminal biotin will be oriented such that the C-terminal portion of the peptide will be distal to the surface.

The present disclosure is further illustrated by the following examples which should not be construed as further limiting. The contents of the figures and all references, patents and published patent applications cited throughout this application are expressly incorporated herein by reference for all purposes.

Furthermore, in accordance with the present disclosure there may be employed conventional molecular biology, microbiology, and recombinant DNA techniques within the skill of the art. Such techniques are explained fully in the literature. See, e.g., Green & Sambrook, Molecular Cloning: A Laboratory Manual, Fourth Edition (2012) Cold Spring Harbor Laboratory Press, Cold Spring Harbor, New York; DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

### EXAMPLES

The following examples are put forth so as to provide those of ordinary skill in the art with a complete disclosure and description of how to make and use the compositions and methods featured in the disclosure, and are not intended to limit the scope of what the Inventors regard as their disclosure. Efforts have been made to ensure accuracy with respect to numbers used (e.g., amounts, temperature, etc.) but some experimental errors and deviations should be accounted for.

Unless indicated otherwise, parts are parts by weight, molecular weight is average molecular weight, temperature is in degrees Centigrade, and pressure is at or near atmospheric.

### Example 1

### Agonistic Mechanism of Action of an Anti-CD40 Monoclonal Antibody

The mechanism of action of a fully human anti-CD40 IgG2 monoclonal antibody (herein termed “AbC1”) was studied. AbC1 is a “pure agonist”, i.e., its binding to CD40 activates the CD40 signaling pathway in an FcγR-independent manner (i.e., independently of Fcγ receptor engagement) or independently of any other type of target-mediated crosslinking. AbC1 comprises a light chain variable region (LCVR) with SEQ ID NO: 1 and a heavy chain variable region (HCVR) with SEQ ID NO: 2.

SEQ ID NO: 1
DIQMTQSPSSVSASVGDRVTITCRASQGIYSWLAWYQQKPGKAPNLLIYTASTLQSGVPSR
FSGSGSGTDFTLTISSLQPEDFATYYCQQANIFPLTFGGGTKVEIK
CDR sequences are highlighted: IMGT numbering in bold; Kabat numbering underlined; Chothia
numbering italicized
SEQ ID NO: 2
QVQLVQSGAEVKKPGASVKVSCKASGYTFTGYYMHWVRQAPGQGLEWMGWINPDSG
GTNYAQKFQGRVTMTRDTSISTAYMELNRLRSDDTAVYYCARDQPLGYCTNGVCSYF
DYWGQGTLVTVSS
CDR sequences are highlighted: IMGT numbering in bold; Kabat numbering underlined; Chothia
numbering italicized

To understand the agonistic mechanism of action of AbC1, we solved the crystal structure of CD40 in complex with the AbC1 Fab. The crystal structure showed hetero-tetramers comprising two copies of CD40 and two copies of AbC1. Beside the cognitive interactions between CD40 and AbC1 in cis, two additional interfaces in the crystal structure were observed: (1) a Fab-CD40 interaction in trans, and (2) a Fab-Fab interface. These additional interactions are typically considered as artefacts due to crystal packing; however, given the agonist property of AbC1, we wondered whether these additional interactions could actually be substantive and contribute to the agonistic mechanism of action of AbC1.

To answer this question, a chemical-crosslinking experiment was performed to examine if this oligomerization state also exists in solution, or if it is an artefact due to crystal packing. After disuccinimidyl suberate (DSS)-mediated crosslinking, a CD40-Fab hetero-tetramer appeared in a concentration-dependent manner, indicating that the observed CD40-Fab hetero-tetramer in the crystal structure also exists in solution.

To test whether these additional interactions could contribute to the pure agonist activity of AbC1, several mutants were designed:
a G4S loop insertion into AbC1 HCVR's CDR1, which creates clashes with the second CD40 molecule in trans;
an engineered glycosylation site at position T28 of the HCVR with SEQ ID NO: 2, which creates clashes with the second CD40 molecule in trans by glycosylation;
mutations at the Fab-CD40 interaction interface in trans (T28A/T30A/Y32F and Y27A/T28A/T30A/Y32F, with reference to SEQ ID NO: 2 numbering);
mutations at the Fab-Fab interaction interface (L102A/Y104A, with reference to SEQ ID NO: 2 numbering).

These mutations were engineered to be on the CD40-Fab trans interface or Fab-Fab interface, so as to not affect the primary CD40 binding site, except for two interface mutants (Y27A/T28A/T30A/Y32F and L102A/Y104A of SEQ ID NO: 2). This was also confirmed by cell-based binding assay. These mutants displayed significantly lower agonistic activity when compared to the parent AbC1 except for the Fab-Fab interface mutant when assayed. Primary CD40 binding was not affected. This result suggests that the secondary Fab-CD40 interaction in trans could be key for the agonistic activity of AbC1.

### Example 2

### Mutant Anti-CD40 Monoclonal Antibodies

Given that the secondary CD40-Fab interface-mediated CD40 clustering would appear to be key for AbC1 agonistic activity, other mutations in this interface were designed and tested to see if these could result in stronger agonist binding proteins.

Through structural analysis, single- and double-point mutations were designed focusing on the Y32 and S77 residues of AbC1's HCVR with SEQ ID NO: 2. All mutants comprised the same LCVR with SEQ ID NO: 1; as well as the same constant regions as AbC1, with SEQ ID NO: 3 for the light chain constant region (LCCR) and SEQ ID NO: 4 for the heavy chain constant region (HCCR).

SEQ ID NO: 3
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 4
ASTKGPSVFPLAPCSRSTSESTAALGCLVKDYFPEPVTVSWNSGALTSGVHTFPAVLQSS
GLYSLSSVVTVPSSNFGTQTYTCNVDHKPSNTKVDKTVERKCCVECPPCPAPPVAGPSV
FLFPPKPKDTLMISRTPEVTCVVVDVSHEDPEVQFNWYVDGVEVHNAKTKPREEQFNST
FRVVSVLTVVHQDWLNGKEYKCKVSNKGLPAPIEKTISKTKGQPREPQVYTLPPSREEM
TKNQVSLTCLVKGFYPSDIAVEWESNGQPENNYKTTPPMLDSDGSFFLYSKLTVDKSRW
QQGNVFSCSVMHEALHNHYTQKSLSLSPGK

The following single and double mutants were produced and tested:
Y32K/S77A, termed “mAb1” in the following;
Y32K/S77P, termed “mAb2” in the following;
Y32K, termed “mAb3” in the following; and
Y32R, termed “mAb4” in the following.

### Example 3

### In Vitro Assays

### Material and Methods

### Surface Plasmon Resonance (SPR) Binding Assay

A Biacore 8K+instrument was used to assess the binding affinity of mutant anti-CD40 mAbs versus parental antibody to recombinant human CD40. Protein A/G was immobilized to the surface of a CM5 series S chip using amine chemistry. Test compounds were then captured to the surface between 120 RU and 170 RU. Recombinant hCD40 was diluted 2-fold from 300 nM to 2.34 nM in HBS-EP+buffer. Association constant (Kₐ) and dissociation constant (K_{d}) were measured for 300 seconds and 450 seconds, respectively. Glycine (10 mM) pH 1.5 was used to regenerate the chip surface. The binding affinity (K_{D}) was determine using a 1:1-binding model by a global fit of all the concentrations.

### Binding Assay

### On Primary Naive Human B-Cells

Goal: evaluate the affinity of mutant anti-CD40 mAbs versus parental antibody AbC1 and isotype control on the receptor expressed by B-cells. Calculate the binding EC₅₀ and the Eₘₐₓ values of the compounds.

Naive B-cell were isolated from PBMC of healthy donors using a negative selection with microbeads (Miltenyi, Cat. 130-091-150) and cultivated in RPMI 1640 medium (ThermoFischer, Cat. 31870-025) supplemented with 10% fetal bovine serum (Biowest, Cat. S181H-100), 1% penicillin/streptomycin (ThermoFischer, Cat. 15140122), 1% sodium pyruvate (ThermoFischer, Cat. 11360) and 1% Glutamax (ThermoFischer, Cat. 35050) using industry standard aseptic techniques.

Naive B-cell were plated at a density of 50×10′ cells per well in 96-well plates and incubated with CD40 agonists, hexameric ligand and anti-CD40 antibodies, for 1 hour at 4° C. Cells were then harvested and incubated with AF 488 conjugated secondary antibody before flow cytometry analysis using a Fortessa X20.

Data from the flow cytometer was analyzed using FlowJo (V10.8.1), then the binding curves, the Eₘₐₓ, and EC₅₀ values were plotted using GraphPad Prism (V9.1.2).

On cynoCD40-expressing HEK293 cells

Goal: evaluate the affinity of mutant anti-CD40 mAbs versus parental antibody AbC1 and isotype control on cynomolgus CD40 receptor and calculate the binding EC₅₀ and the Eₘₐₓ values of the compounds.

The binding assay was performed using in-house transfected HEK293 cells expressing cyno CD40. Cyno CD40-expressing HEK293 cells were cultivated in DMEM (ThermoFischer, Cat. 31966) supplemented with 10% fetal bovine serum (Biowest, Cat. S181H-100), 1 mg/mL geneticin (ThermoFischer, Cat. 10131035) using industry standard aseptic techniques.

Cyno CD40-expressing HEK293 cells were plated at a density of 50×10³ cells per well in 96-well plates and incubated with serially diluted concentrations of CD40 agonists, hexameric ligand and anti-CD40 antibodies, for 1 hour at 4° C. Cells were then harvested and incubated with AF488-conjugated secondary antibody before flow cytometry analysis using a Fortessa X20.

Data from the flow cytometer was analyzed using FlowJo (V10.8.1), then the binding curves, the Eₘₐₓ, and EC₅₀ values were plotted using GraphPad Prism (V9.1.2).

### Reporter Assay

### On Human CD40 Reporter Cells

Goal: assess the activity of mutant anti-CD40 mAbs versus parental antibody AbC1 and isotype control and calculate the EC₅₀ and the Eₘₐₓ for each of the compounds.

NFkB-Luc2P/U-2 OS cells (Promega, Cat. J2132) were grown in McCoy's 5A (ThermoFischer, Cat. 26600) supplemented with 10% fetal bovine serum (Biowest, Cat. S181H-100), 1% penicillin/streptomycin (ThermoFischer, Cat. 15140122) and 1/250 hygromycin (ThermoFischer, Cat. 10687010) using industry standard aseptic techniques.

NFkB-Luc2P/U-2 OS cells were plated at a density of 25×10³ cells per well in 96-well plate white flat bottom culture plates and were allowed to proliferate overnight at 37° C. in RPMI 1640 medium (ThermoFischer, Cat. 31870-025) supplemented with 1% fetal bovine serum (Biowest, Cat. S181H-100). The following day, the medium was removed and serially diluted concentrations of CD40 agonists or isotypes were added on the cell culture. After an incubation of 4 hours at 37° C., Bio-Glo Reagent (Promega, Cat. G7941) was added to each well containing cells. Luminescence was then measured using a GloMax® Discover plate reader.

The binding curves, Eₘₐₓ, and EC50 values were plotted using GraphPad Prism (V9.1.2).

### On Rhesus CD40 Reporter Cells

Goal: assess the activity of mutant anti-CD40 mAbs versus parental antibody AbC1 and isotype control in rhesus CD40 reporter cells.

The reporter assay was performed using in-house transfected HEK293 cells expressing rhesus CD40. HEK293H rhesus CD40 reporter cells were cultivated in DMEM (ThermoFischer, Cat. 31966) supplemented with 10% fetal bovine serum (Biowest, Cat. S181H-100), 200 pg/mL hygromycin B (ThermoFischer, Cat. 10687010) and 100 pg/mL zeocin (ThermoFischer, Cat. R25001) using industry standard aseptic techniques.

HEK293H rhesus CD40 reporter cells were plated at a density of 25×10³ cells per well in 96-well plate white flat bottom culture plates and were allowed to proliferate overnight at 37° C. in DMEM (ThermoFischer, Cat. 31966) supplemented with 10% fetal bovine serum (Biowest, Cat. S181H-100). Serially diluted concentrations of CD40 agonists or isotypes were added on the cell culture. After an overnight incubation at 37° C., Bio-Glo reagent (Promega, Cat. G7941) was added to each well containing cells. Luminescence was then measured using a GloMax® Discover plate reader.

The binding curves, Eₘₐₓ, and EC₅₀ values were plotted using GraphPad Prism (V9.1.2).

### Human B-Cell Activation Assay (BCA)

Goal: assess the activity of mutant anti-CD40 mAbs versus parental antibody AbC1 and isotype control in human B-cells. Observe the B-cell activation markers and co-stimulatory molecules.

CD20+B-cell were isolated from PBMCs of healthy donors using CD20 microbeads (Miltenyi, Cat. 130-091-104) and cultivated in RPMI 1640 medium (ThermoFischer, Cat. 31870-025) supplemented with 10% fetal bovine serum (Biowest, Cat. S181H-100), 1% penicillin/streptomycin (ThermoFischer, Cat. 15140122), 1% sodium pyruvate (ThermoFischer, Cat. 11360) and 1% Glutamax (ThermoFischer, Cat. 35050) using industry standard aseptic techniques.

CD20+B-cell were plated at a density of 150×10³ cells per well in 96-well plates and incubated with CD40 agonists, hexameric ligand and anti-CD40 antibodies, for 68 hours at 37° C. in humidified conditions. Cells were then harvested for flow cytometry analysis of B-cell activation markers and costimulatory molecules such as CD69, CD86 and CD267 (TACI; Transmembrane Activator and Calcium-modulator and CAML Interactor) using a Fortessa X20.

Flow cytometry results were analyzed using FlowJo (V10.8.1), then plotted using GraphPad Prism (V9.1.2).

### Monocyte-Derived Dendritic Cell (MoDC) Maturation Assay

Goal: assess the activity of mutant anti-CD40 mAbs versus parental antibody AbC1 and isotype control on MoDC activation and maturation markers.

CD14+monocytes were isolated from PBMCs of healthy donors using CD14 microbeads (Miltenyi, Cat. 130-050-201) and cultivated in RPMI 1640 medium (ThermoFischer, Cat. 31870-025) supplemented with 10% fetal bovine serum (Biowest, Cat. S181H-100), 1% penicillin/streptomycin (ThermoFischer, Cat. 15140122) and 1% Glutamax (ThermoFischer, Cat. 35050) using industry standard aseptic techniques. Cells were differentiated in immature MoDC by incubated CD14⁺ cells in presence of GM-CSF (50 ng/mL, Miltenyi, Cat. 130-093-867) and IL-4 (10 ng/mL, Miltenyi, Cat. 130-093-922) for 5 days at 37° C. in humidified conditions.

Immature MoDC were plated at a density of 200×10³ cells per well in 96-well plates and incubated with CD40 agonists, hexameric ligand and anti-CD40 antibodies, for 24 hours at 37° C. in humidified conditions. Cells were then harvested for flow cytometry analysis of MoDC maturation markers such as CD80, CD83 and CD86 using a Fortessa X20.

Flow cytometry results were analyzed using FlowJo (V10.8.1), then plotted using GraphPad Prism (V9.1.2).

### Results

### Surface Plasmon Resonance (SPR) Binding Assay

The results of the SPR binding assay are given in Table 1.

TABLE 1

| Test compound | Kₐ (M⁻¹s⁻¹) | K_{d} (s⁻¹) | K_{D} (M) |
|---|---|---|---|
| AbC1 | 1.1 × 10⁵ | 1.9 × 10⁻³ | 1.8 × 10⁻⁸ |
| mAb1 | 5.2 × 10⁴ | 3.4 × 10⁻³ | 6.5 × 10⁻⁸ |
| mAb3 | 4.5 × 10⁴ | 4.1 × 10⁻³ | 9.1 × 10⁻⁸ |

### Binding Assay

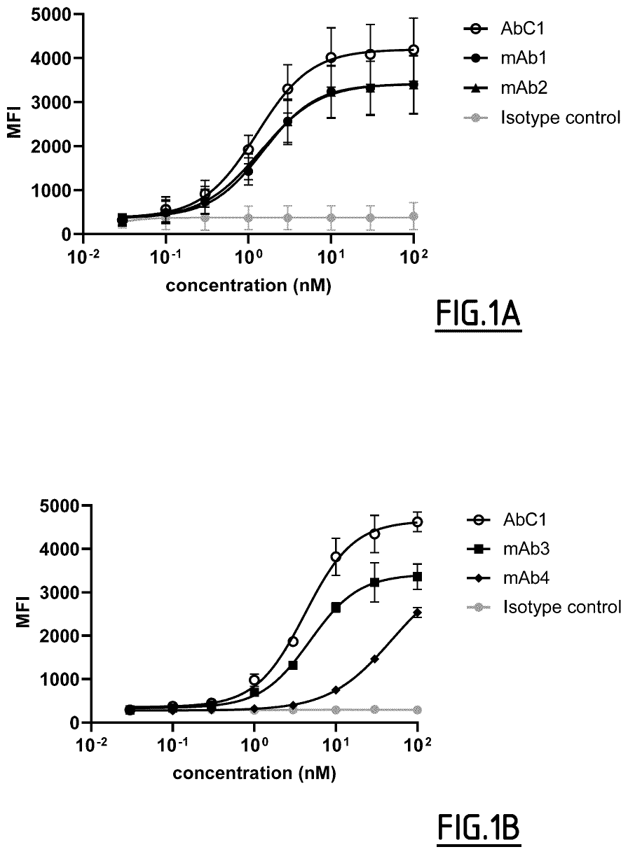

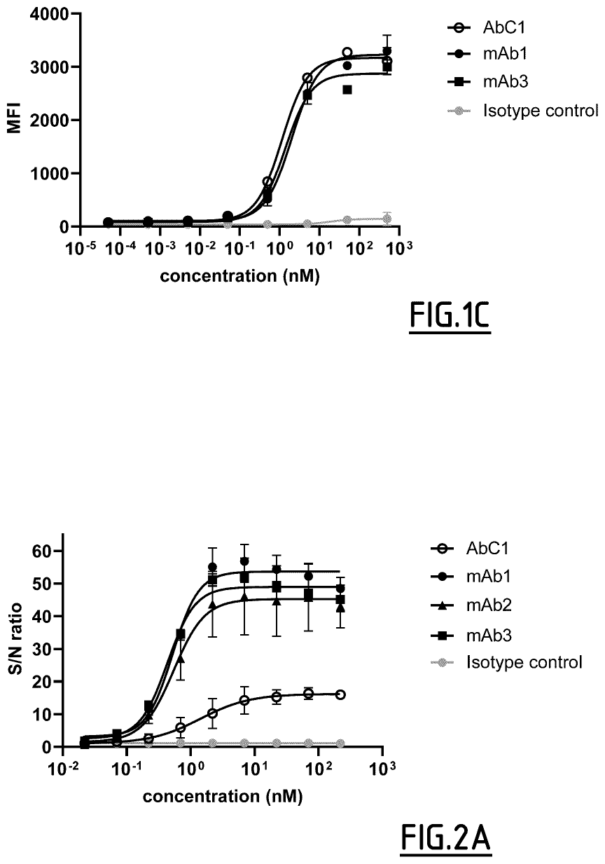

Binding assays on human primary naive B-cells and on cynomolgus CD40-expressing HEK293 cells showed that the binding to hCD40 (FIGS. 1A-B) and cyno CD40 (FIG. 1C) is preserved with each of mAb1-mAb4 compared to the parental antibody AbC1.

### Reporter Assay

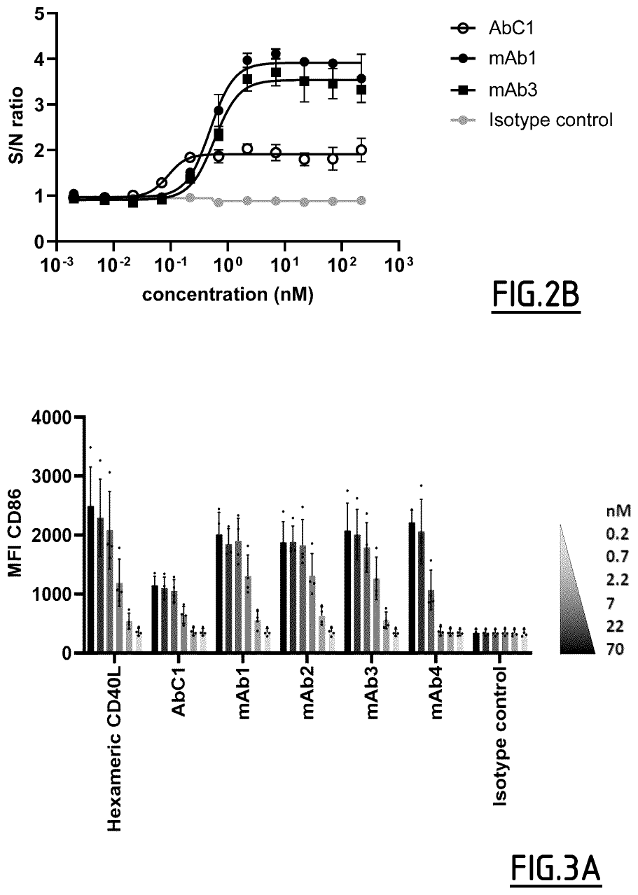

The result showed that the single mutation Y32K in mAb3 increased the agonistic activity of the mutant anti-CD40 mAb in human CD40 U-2 OS reporter cells (FIG. 2A; Table 2) and in rhesus CD40 reporter cells (FIG. 2B; Table 2). A combination of the Y32K with 577A or S77P in mAb1 and mAb2 respectively showed a slight improvement of the activity compared to the single Y32K mutation alone, although not statistically significant.

TABLE 2

| | Human | | Rhesus | |
|---|---|---|---|---|
| | EC₅₀ (nM) | Top (nM) | EC₅₀ (nM) | Top (nM) |
| AbC1 | 1.43 | 16.20 | 0.09 | 1.91 |
| mAb1 | 0.52 | 53.65 | 0.49 | 3.91 |
| mAb2 | 0.54 | 45.23 | n.d. | n.d. |
| mAb3 | 0.44 | 48.94 | 0.59 | 3.54 |

### B-Cell Activation Assay (BCA)

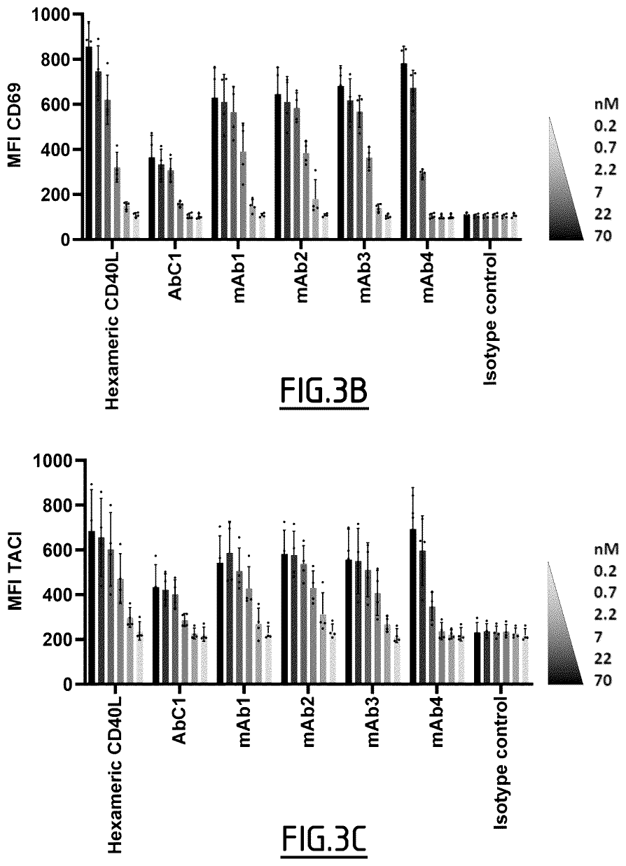

In a primary B-cell activation assay, all four mutants mAb1-4 equaled CD40's natural ligand (hexameric CD40L) control activity, and demonstrated a superior activity compared to the parental AbC1 antibody (FIGS. 3A-C).

### Monocyte-Derived Dendritic Cell (MoDC) Maturation Assay

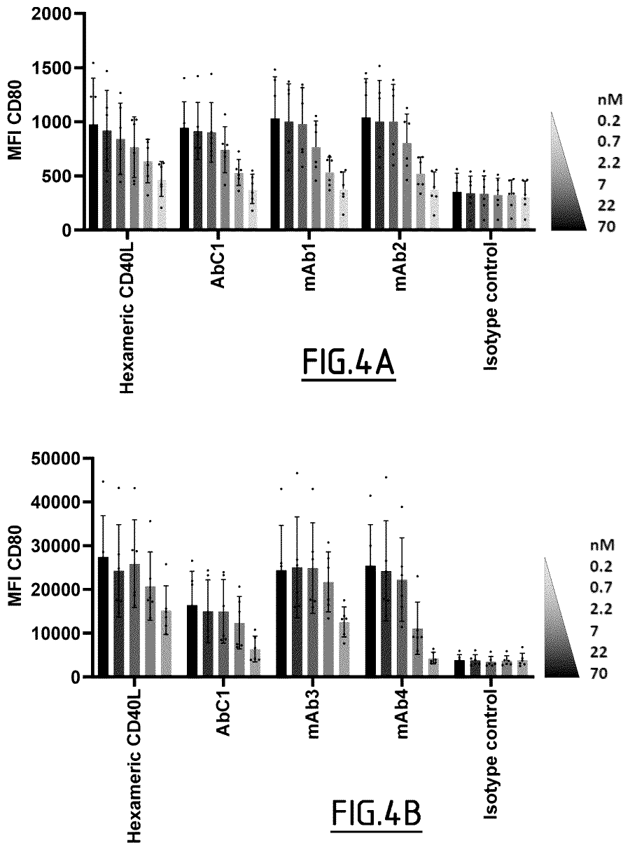

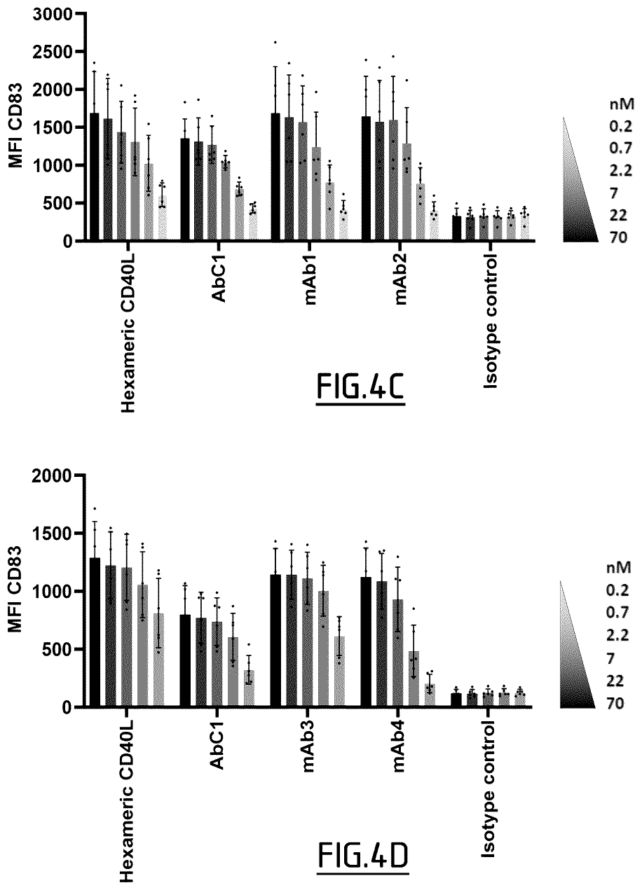

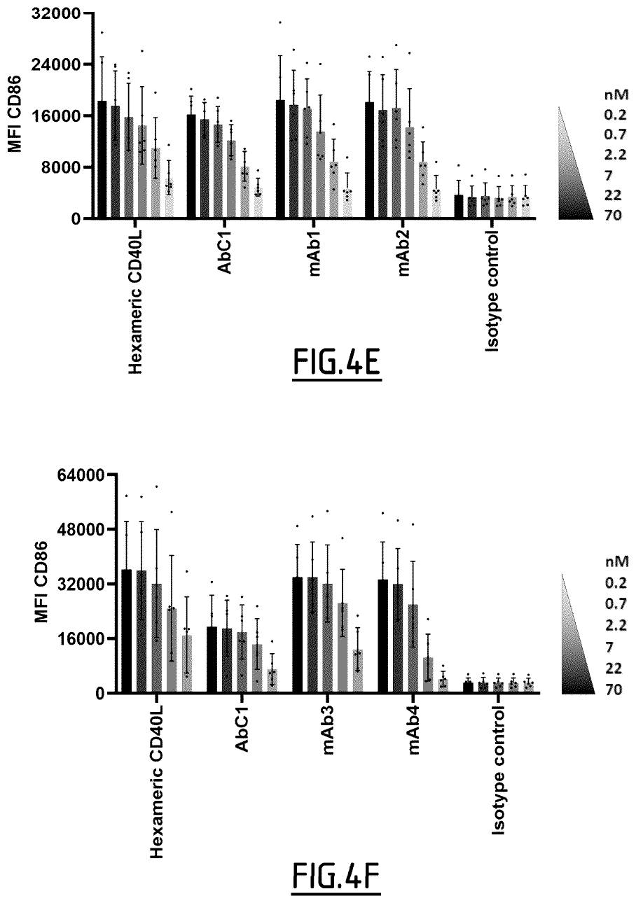

Finally, in a primary monocyte-derived dendritic cell (MoDC) assay, all four mutants mAb1-4 equaled the hexameric CD40L control activity, and again demonstrated a superior activity compared to the parental AbC1 antibody (FIGS. 4A-F).

### Example 4

### Conditionally-Active Anti-CD40 Monoclonal Antibodies

We generated conditionally-active anti-CD40 monoclonal antibodies from the “mAb1” double mutant (Y32K/S77A) described in Example 2.

Briefly, the conditionally-active anti-CD40 monoclonal antibodies comprise:
a “masked” light chain, including from N- to C-terminal, a masking moiety (MM), a cleavable linker (CL) and the anti-CD40 “mAb1” light chain (LCVR with SEQ ID NO: 1 and LCCR with SEQ ID NO: 3); and
the anti-CD40 “mAb1” heavy chain (HCVR with SEQ ID NO: 5 and HCCR with SEQ ID NO: 4).

Cleavable linkers typically comprise a short amino acid sequence which is the target for a protease. In the case of cancer treatment, it is preferable that the protease be a tumor-specific protease, i.e., a protease which is, if not exclusively, at least predominantly found in the tumor microenvironment in vivo. Hence, in absence of protease, the masking moiety remains fused to the antibody, thereby reducing, inhibiting or abrogating the binding of the antibody to its target (here, to CD40). However, when the conditionally-active antibody colocalizes with a protease capable of cleaving the cleavable linker, e.g., in a tumor microenvironment, the masking moiety is released from the antibody and binding of the latter to the target antigen (e.g., CD40) is restored.

Two alternative masking moieties (hereafter, “MM1” and “MM2”) were identified following Adagene's protocol using a synthetic library as described in International application publication WO 2019/149282 A1. MM1 or MM2 was linked in N-terminus of the LCVR of “mAb1” through one of two alternative cleavable linkers (CL1 or CL2). Combining one masking moiety with one cleavable linker led to four different conditionally-active (masked) anti-CD40 monoclonal antibodies:
MC1, comprising MM1 and CL1;
MC2, comprising MM2 and CL1;
MC3, comprising MM1 and CL2; and
MC4, comprising MM2 and CL2.

### Material and Methods

### MMP9-Mediated Cleavage

For experiments requiring cleavage of a masking moiety by MMP9 protease (compounds identified as “MMP9-activated” hereafter).

In a first step, recombinant human MMP9 protein (R&D Systems, Ref. 911-MP-010) was activated using p-aminophenylmercuric acetate (APMA; Calbiochem, Ref. 164610-700MG), added to a final concentration of 1 mM into MMP9 solution at 100 μg/mL and incubated at 37° C. for 24 hours. Activated MMP9 was then aliquoted and stored at −80° C. until further use.

Then, test (masked) compounds were diluted to 1 mg/mL and incubated for 24 hours at 37° C. under gentle shake (300 rpm) with activated MMP9 in a final concentration of 5 nM.

If desired, to remove excess masking peptide, the samples were purified using using a HiLoad® 26/600 Superdex® 200 (GE Healthcare, Ref. 28-9893-36).

uPA-Mediated Cleavage

For experiments requiring cleavage of a masking moiety by uPA protease (compounds identified as “uPA-activated” hereafter).

Test (masked) compounds were diluted to 1 mg/mL and incubated for 24 hours at 37° C. under gentle shake (300 rpm) with recombinant human uPA protein (Sino Biological, Ref. 10815-H08H-A) in a final concentration of 209 nM.

If desired, to remove excess masking peptide, the samples were purified using a HiLoad® 16/600 Superdex® 200 (GE Healthcare, Ref. 28-9893-35).

### Binding Assay

### On Raji Cells

The dose-dependent binding activity on Raji cells (human B lymphoblastoid cells originally derived from a patient with Burkitt lymphoma) of the mAb1 double mutant (without masking moiety), of each of MC1-MC4 (masked), and of protease-activated compounds (de-masked) was measured using flow cytometry.

Briefly, Raji cells were seeded in 96-well plates at 1.0×10⁵ cells/well and incubated with serially-diluted test antibodies for 60 minutes at 4° C. in 2% FBS/dPBS buffer. The cells were then washed twice with dPBS and further incubated with secondary allophycocyanin (APC)-conjugated AffiniPure F(ab′)₂ fragment donkey anti-human IgG (H+L) antibody (Jackson ImmunoResearch, Ref. 709-136-149) (1:500) for 30 minutes at 4° C. Finally, the cells were washed twice with dPBS and suspended in FACS buffer for flow cytometry analysis. MFI values vs concentrations were analyzed by FlowJo and data were further fitted with four-parameter non-linear regression to get EC₅₀ values by GraphPad Prism software.

On U-2 OS/NFκB cells

The dose-dependent binding activity on U-2 OS/NFκB cells of the mAb1 double mutant (without masking moiety), of each of MC1-MC4 (masked), and of protease-activated compounds (de-masked) was measured using flow cytometry.

Briefly, cultured U-2 OS/NFκB cells were seeded in 96-well plates at 1.0×10⁵ cells/well and incubated with serially-diluted test antibodies for 60 minutes at 4° C. in 2% FBS/dPBS buffer. The cells were then washed twice with dPBS and further incubated with secondary allophycocyanin (APC)-conjugated AffiniPure F(ab′)₂ fragment donkey anti-human IgG (H+L) antibody (Jackson ImmunoResearch, Ref. 709-136-149) (1:500) for 30 minutes at 4° C. Finally, the cells were washed twice with dPBS and suspended in FACS buffer for flow cytometry analysis. MFI values vs concentrations were analyzed by FlowJo and data were further fitted with four-parameter non-linear regression to get EC₅₀ values by GraphPad Prism software.

### Reporter Assay

The mAb1 double mutant (without masking moiety), each of MC1-MC4 (masked), and protease-activated compounds (de-masked) were characterized by a luciferase-based reporter assay.

Briefly, U-2 OS/NFκB cells (5×10⁴ cells/well in 96-well plate), which contain a luciferase gene under the control of the NFκB response element, were mixed with serial dilutions of test antibodies. After incubation for about 5 hours at 37° C. and 5% CO₂, ONE-GLO substrate was added, and the luminescence was measured by a multiplate reader (Molecular Devices SpectraMax i3x).

### B-Cell Activation Assay

### Human

The in vitro biological activity of the mAb1 double mutant (without masking moiety), each of MC1-MC4 (masked), and protease-activated compounds (de-masked) was measured on primary human B cells using flow cytometry.

Briefly, total human B cells were purified from human peripheral blood mononuclear cells (PBMCs) (D #A10K797075) with StemCell kit (17954). These cells (5×10⁴ cells/well in 96-well plates) were incubated with titrated test antibodies for 2 days. CD86 (detected with anti-CD86, BioLegend, 1:200 dilution) expression on human B-cells was assessed by flow cytometry.

### Mouse

Goal: Assess the activity of mutant anti-CD40 mAbs versus parental antibody AbC1 and isotype control in murine B-cells. Observe the expression of key activation markers and the cytokine release from activated murine B cells.

hCD40 KI mice from CIPHE (Center for Immunophenomics, Marseille, France) were used to perform the experiments. Murine B-cells were isolated from splenocytes via a negative selection. B-cells were plated at a density of 150×10³ cells/well in 96-flat bottom well plates and cultured in complete media comprising RPMI 1640 (Gibco 31870-025), 2 mM L-glutamine (Gibco 25030-081), 10% FCS (Eurobio CVFSFO0-01), 1× non-essential amino acids (Gibco 11140-050), 1 mM sodium pyruvate (Gibco 11360-070), 0.05 mM 2-mercaptoethanol (Gibco 31350-010), supplemented with 1% penicillin/streptomycin.

Serially diluted concentrations of test compounds were added to the culture. After 2 days of incubation, the expression of key activation markers and cytokine secretion were evaluated in the harvested supernatants using a mouse inflammation cytometric bead array (CBA) kit on a flow cytometer according to manufacturer's instructions (BD Bioscience 552364).

The expression of the activation markers was analyzed with FlowJo (V10.9.0), then plotted using GraphPad Prism (V9.5.0). The cytokines secretion was analyzed with FCAP array (V3.0.19.2091) for CBA samples, then plotted using GraphPad Prism.

### Monocyte-Derived Dendritic Cell (MoDC) Maturation Assay

For MoDC maturation assays, see protocol in Example 3.

### Results

### Binding Assay

### On Raji Cells

As shown in Table 3 and FIG. 5, the protease-activated (i.e., where the masking moiety is cleaved) compounds (MMP9-activated and uPA-activated) exhibit similar binding activity to Raji cells compared to the mAb1 double mutant (without masking moiety) based on EC₅₀ values. Binding is reduced by at least 107-fold (for MC4) by EC₅₀ comparison for all masked compounds MC1-MC4 relative to the mAb1 double mutant, and up to 364-fold (for MC2).

TABLE 3

| | | Relative masking |
|---|---|---|
| Test antibodies | EC₅₀ (nM) | efficacy |
| mAb1 (without masking moiety) | 1.34 | 1 |
| MC1 | 475.7 | 355 |
| MC2 | 487.6 | 364 |
| MC3 | 387.7 | 289 |
| MC4 | 143.1 | 107 |
| MMP9-activated (MC1/MC2) | 1.464 | 1.1 |
| uPA-activated (MC3/MC4) | 2.006 | 1.5 |

### On U-2 OS/NFκB Cells

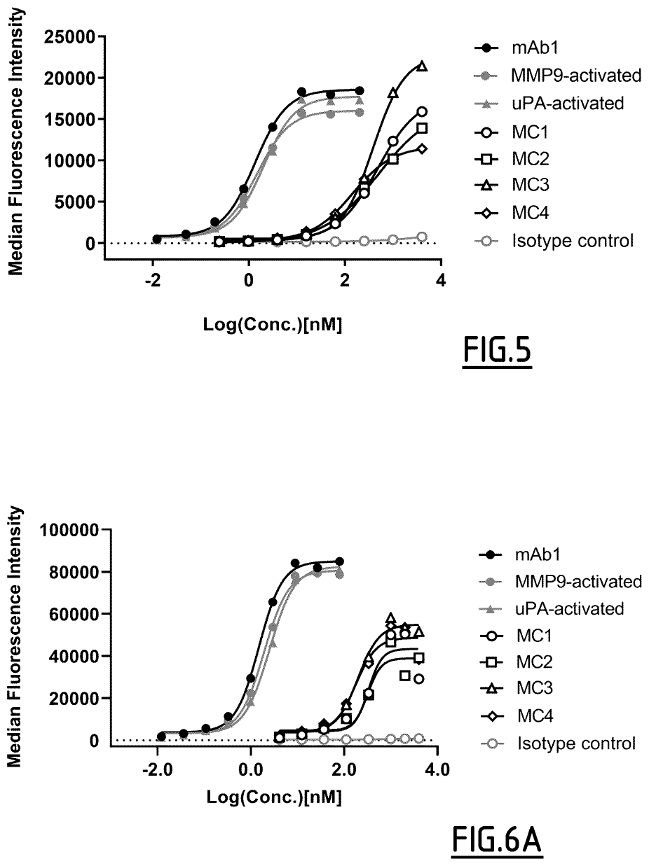

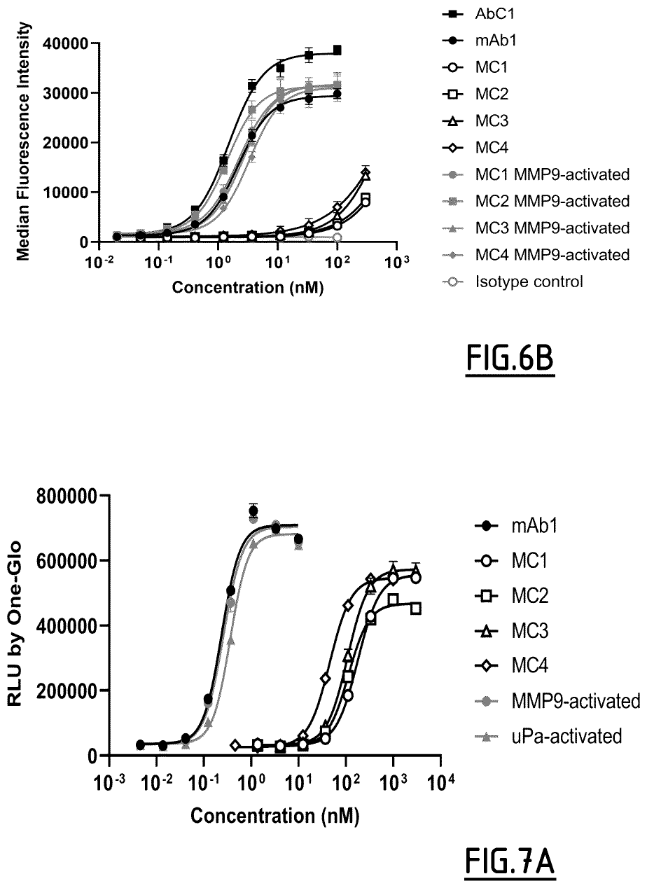

As shown in Table 4 and FIG. 6A, the protease-activated (i.e., where the masking moiety is cleaved) compounds (MMP9-activated and uPA-activated) exhibit similar binding activity to U-2 OS/NFκB cells compared to the mAb1 double mutant (without masking moiety) based on EC₅₀ values. Binding is reduced by at least 118-fold (for MC4) by EC₅₀ comparison for all masked compounds MC1-MC4 relative to the mAb1 double mutant, and up to 220-fold (for MC1).

TABLE 4

| | | Relative masking |
|---|---|---|
| Test antibodies | EC₅₀ (nM) | efficacy |
| mAb1 (without masking moiety) | 1.499 | 1 |
| MC1 | 330.1 | 220 |
| MC2 | 316.8 | 211 |
| MC3 | 196.9 | 131 |
| MC4 | 176.4 | 118 |
| MMP9-activated (MC1/MC2) | 1.936 | 1.3 |
| uPA-activated (MC3/MC4) | 2.515 | 1.7 |

In a further experiment on U-2 OS cells, mAb1 (unmasked) showed a comparable EC₅₀ towards CD40 to that of the parental AbC1 antibody. All four conditionally-active anti-CD40 antibodies tested had similar EC₅₀ towards CD40. After MMP9-mediated cleavage of their masking moiety, all four demasked antibodies retrieved an EC₅₀ equivalent to that of mAb1 (unmasked) and comparable to that of the parental AbC1 antibody (FIG. 6B).

### Reporter Assay

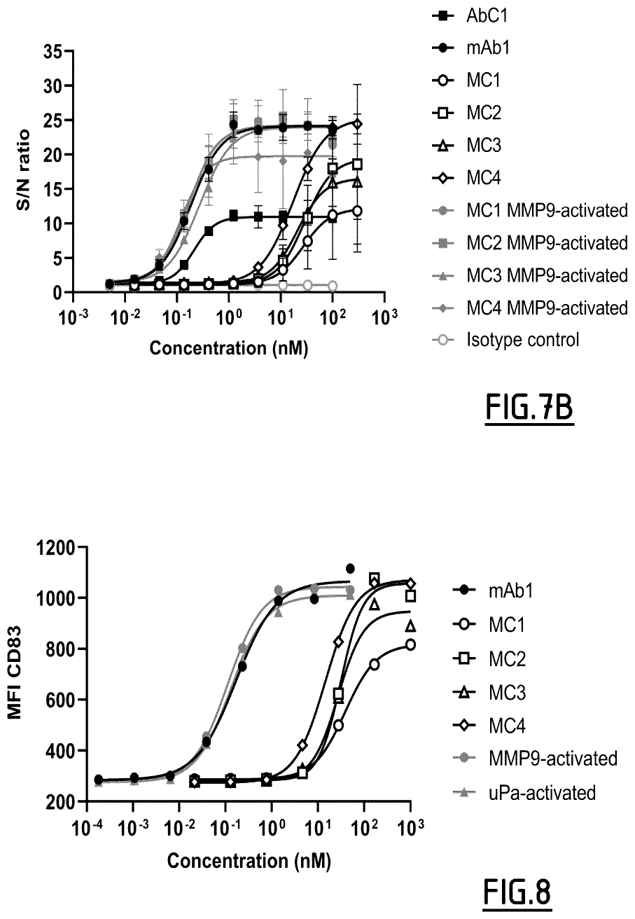

As shown in Table 5 and FIG. 7A, the protease-activated (i.e., where the masking moiety is cleaved) compounds (MMP9-activated and uPA-activated) exhibit similar reporter gene activities compared to the mAb1 double mutant (without masking moiety) based on EC₅₀ values and maximal signals. EC₅₀ values are shifted relative to the mAb1 double mutant (without masking moiety) by at least 193-fold (for MC4) with masked compounds MC1-MC4 relative to the mAb1 double mutant, and up to 753-fold (for MC1).

TABLE 5

| | | Relative masking |
|---|---|---|
| Test antibodies | EC₅₀ (nM) | efficacy |
| mAb1 (without masking moiety) | 0.2376 | 1 |
| MC1 | 178.9 | 753 |
| MC2 | 113.3 | 477 |
| MC3 | 108.5 | 457 |
| MC4 | 45.81 | 193 |
| MMP9-activated (MC1/MC2) | 0.2606 | 1.1 |
| uPA-activated (MC3/MC4) | 0.3625 | 1.5 |

In a further experiment, mAb1 (unmasked) showed a clear activity superior to that of the parental AbC1 antibody. The conditionally-active anti-CD40 antibodies with cleavable linker CL1 (MC1 and MC2) had an EC₅₀ higher in the CD40 reporter assay than those with cleavable linker CL2 (MC3 and MC4), although in an equivalent nM range. After MMP9-mediated cleavage of their masking moiety, all four demasked antibodies retrieved an EC₅₀ equivalent to that of mAb1 (unmasked) (FIG. 7B).

### B-Cell Activation Assay

### Human

As shown in Table 6 and FIG. 8, the protease-activated (i.e., where the masking moiety is cleaved) compounds (MMP9-activated and uPA-activated) exhibit similar B-cell activation activities compared to the mAb1 double mutant (without masking moiety) based on EC₅₀ values and maximal CD86 signals. EC₅₀ values are shifted relative to the mAb1 double mutant (without masking moiety) by at least 84-fold (for MC4) with masked compounds MC1-MC4 relative to the mAb1 double mutant, and up to 230-fold (for MC1).

TABLE 6

| | | Relative masking |
|---|---|---|
| Test antibodies | EC₅₀ (nM) | efficacy |
| mAb1 (without masking moiety) | 0.1747 | 1 |
| MC1 | 40.34 | 230.91 |
| MC2 | 28.49 | 176.07 |
| MC3 | 27.86 | 157.76 |
| MC4 | 14.68 | 84.03 |
| MMP9-activated (MC1/MC2) | 0.1144 | 0.65 |
| uPA-activated (MC3/MC4) | 0.1343 | 0.77 |

### Mouse

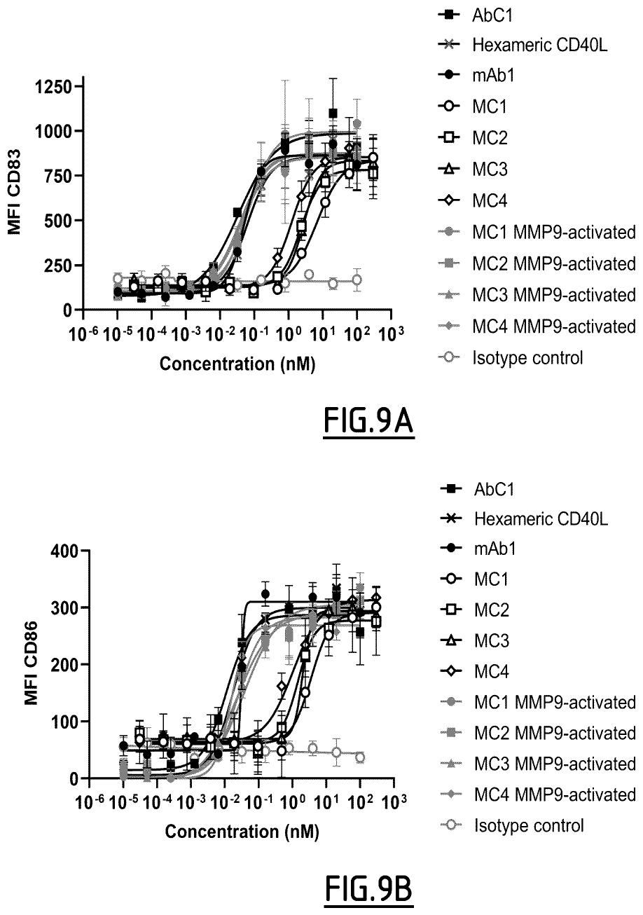

As shown in Table 7 and FIGS. 9A-9B, the protease-activated (i.e., where the masking moiety is cleaved) compounds (MMP9-activated) exhibit similar activation activities in murine B-cells compared to the mAb1 double mutant (without masking moiety) based on EC₅₀ values and maximal CD83 and CD86 signals.

TABLE 7

| | CD83 | | CD86 | |
|---|---|---|---|---|
| | | Relative | | Relative |
| | EC₅₀ | masking | EC₅₀ | masking |
| Test antibodies | (nM) | efficacy | (nM) | efficacy |
| AbC1 | 0.034 | n/a | 0.011 | n/a |
| mAb1 (without masking | 0.051 | 1 | 0.031 | 1 |
| moiety) | | | | |
| MC1 | 6.703 | 131.43 | 3.572 | 115.23 |
| MC2 | 2.308 | 45.25 | 1.462 | 47.16 |
| MC3 | 2.841 | 55.71 | 2.012 | 64.90 |
| MC4 | 1.239 | 24.29 | 1.049 | 33.84 |
| MC1 MMP9-activated | 0.069 | 1.35 | 0.038 | 1.23 |
| MC2 MMP9-activated | 0.048 | 0.94 | 0.024 | 0.77 |
| MC3 MMP9-activated | 0.038 | 0.75 | 0.023 | 0.74 |
| MC4 MMP9-activated | 0.040 | 0.78 | 0.015 | 0.48 |

### Monocyte-Derived Dendritic Cell (MoDC) Maturation Assay

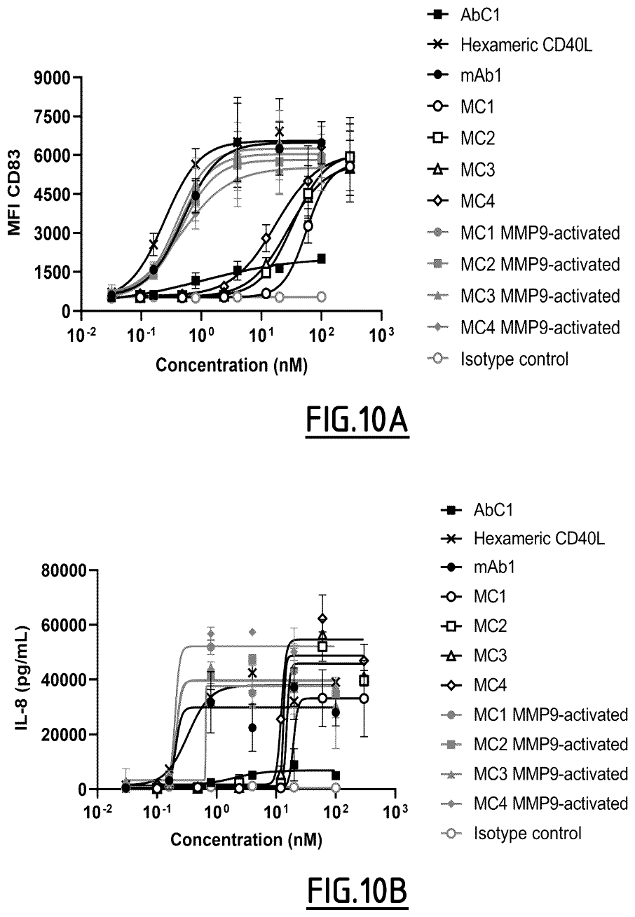

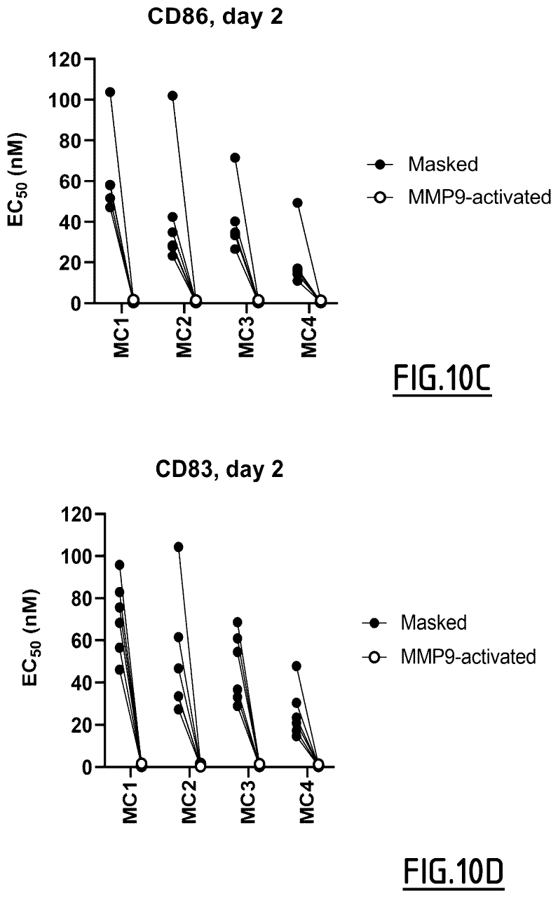

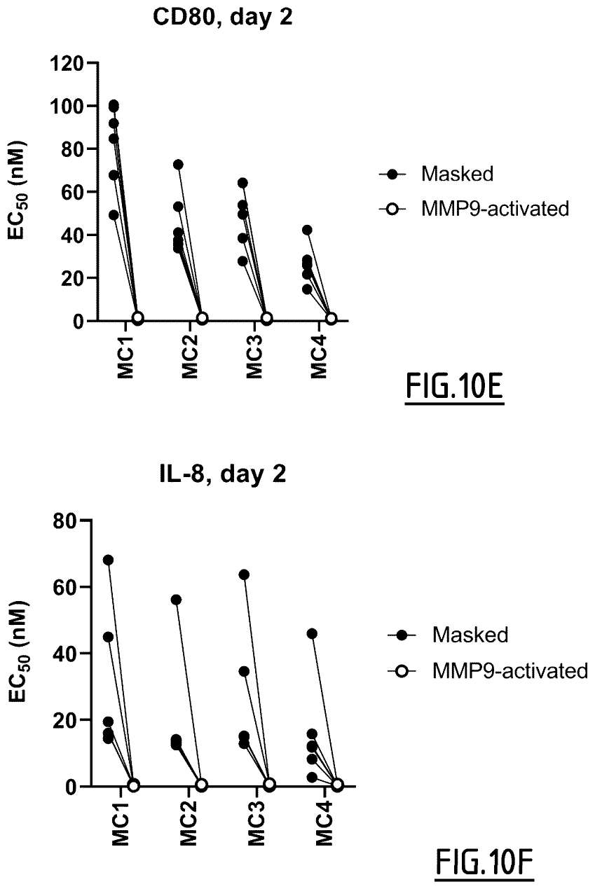

All test compounds induced the secretion of CD83 and IL-8 with a dose-dependent effect (FIGS. 10A-10B). The mAb1 double mutant (without masking moiety) exhibited better EC₅₀ than the parental antibody AbC1, and close to that of CD40's natural ligand, hexameric CD40L.

After MMP9-mediated cleavage of their masking moiety, all four demasked antibodies were more active than their masked counterparts MC1-MC4, with an EC₅₀ similar to that of mAb1 (unmasked) (Table 8; EC₅₀ in nM). Overall, we observed a donor- and cytokine-dependent masking shift (CD86: FIG. 10C; CD83: FIG. 10D; CD80: FIG. 10E; IL-8: FIG. 10F).

TABLE 8

| Test articles | CD83 | IL-8 |
|---|---|---|
| Hexameric CD40L | 0.24 | 0.33 |
| AbC1 | 1.02 | 1.92 |
| mAb1 (unmasked) | 0.49 | 0.21 |
| MC1 | 56.55 | 19.53 |
| MC1 MMP9-activated | 0.41 | 0.21 |
| MC2 | 33.13 | 15.11 |
| MC2 MMP9-activated | 0.46 | 0.21 |
| MC3 | 27.38 | 13.26 |
| MC3 MMP9-activated | 0.40 | 0.66 |
| MC4 | 17.22 | 12.22 |
| MC4 MMP9-activated | 0.45 | 0.20 |

### Sequences

All mutant anti-CD40 mAbs described herein share the same parental LCVR with SEQ ID NO: 1.

The HCVR of “mAb1” (Y32K/S77A) comprises or consists of the amino acid sequence with SEQ ID NO: 5.

SEQ ID NO: 5
QVQLVQSGAEVKKPGASVKVSCKASGYTFTGKYMHWVRQAPGQGLEWMGWINPDSG
GTNYAQKFQGRVTMTRDTSIATAYMELNRLRSDDTAVYYCARDQPLGYCTNGVCSYF
DYWGQGTLVTVSS
CDR sequences are highlighted: IMGT numbering in bold; Kabat numbering underlined;
Chothia numbering italicized

The HCVR of “mAb2” (Y32K/S77P) comprises or consists of the amino acid sequence with SEQ ID NO: 6.

SEQ ID NO: 6
QVQLVQSGAEVKKPGASVKVSCKASGYTFTGKYMHWVRQAPGQGLEWMGWINPDSG
GTNYAQKFQGRVTMTRDTSIPTAYMELNRLRSDDTAVYYCARDQPLGYCTNGVCSYF
DYWGQGTLVTVSS
CDR sequences are highlighted: IMGT numbering in bold; Kabat numbering underlined;
Chothia numbering italicized

The HCVR of “mAb3” (Y32K) comprises or consists of the amino acid sequence with SEQ ID NO: 7′

SEQ ID NO: 7
QVQLVQSGAEVKKPGASVKVSCKASGYTFTGKYMHWVRQAPGQGLEWMGWINPDSG
GTNYAQKFQGRVTMTRDTSISTAYMELNRLRSDDTAVYYCARDQPLGYCTNGVCSYF
DYWGQGTLVTVSS
CDR sequences are highlighted: IMGT numbering in bold; Kabat numbering underlined;
Chothia numbering italicized

The HCVR of “mAb4” (Y32R) comprises or consists of the amino acid sequence with SEQ ID NO: 8.

SEQ ID NO: 8
QVQLVQSGAEVKKPGASVKVSCKASGYTFTGRYMHWVRQAPGQGLEWMGWINPDSG
GTNYAQKFQGRVTMTRDTSISTAYMELNRLRSDDTAVYYCARDQPLGYCTNGVCSYF
DYWGQGTLVTVSS
CDR sequences are highlighted: IMGT numbering in bold; Kabat numbering underlined;
Chothia numbering italicized

The masking moiety #1 (MM1) has an amino acid sequence set forth as

(SEQ ID NO: 22)
EDEPGIRCYYNDDTCQY.

The masking moiety #2 (MM2) has an amino acid sequence set forth as

(SEQ ID NO: 23)
EVGSYYVCPVRFQYFCEE.

Cleavable linker #1 (CL1) has an amino acid sequence set forth as: wherein PLGLAG (in bold; SEQ ID NO: 38) is a matrix metalloproteinase (MMP) substrate.

Cleavable linker #2 (CL2) has an amino acid sequence set format DNA

(SEQ ID NO: 24)
GGGPLGLAGSGGS,

wherein SGRSA (underlined; SEQ ID NO: 39) is a urokinase-type plasminogen activator (uPa) substrate, and PLGLAG (in bold; SEQ ID NO: 38) is a matrix metalloproteinase (MMP) substrate.

(SEQ ID NO: 25)
SGRSAGGGGPLGLAGSGGS,

The LCVR of “MC1” comprises or consists of the amino acid sequence with SEQ ID NO: 26.

SEQ ID NO: 26
EDEPGIRCYYNDDTCQYGGGPLGLAGSGGSDIQMTQSPSSVSASVGDRVTITCRASQGIY
SWLAWYQQKPGKAPNLLIYTASTLQSGVPSRFSGSGSGTDFTLTISSLQPEDFATYYCQQ
ANIFPLTFGGGTKVEIK
CDR sequences are highlighted: IMGT numbering in bold; Kabat numbering underlined;
Chothia numbering italicized

The LCVR of “MC2” comprises or consists of the amino acid sequence with SEQ ID NO: 27.

SEQ ID NO: 27
EVGSYYVCPVRFQYFCEEGGGPLGLAGSGGSDIQMTQSPSSVSASVGDRVTITCRASQGI
YSWLAWYQQKPGKAPNLLIYTASTLQSGVPSRFSGSGSGTDFTLTISSLQPEDFATYYCQQ
ANIFPLTFGGGTKVEIK
CDR sequences are highlighted: IMGT numbering in bold; Kabat numbering underlined;
Chothia numbering italicized

The LCVR of “MC3” comprises or consists of the amino acid sequence with SEQ ID NO: 28.

SEQ ID NO: 28
EDEPGIRCYYNDDTCQYSGRSAGGGGPLGLAGSGGSDIQMTQSPSSVSASVGDRVTITC
RASQGIYSWLAWYQQKPGKAPNLLIYTASTLQSGVPSRFSGSGSGTDFTLTISSLQPEDFAT
YYCQQANIFPLTFGGGTKVEIK
CDR sequences are highlighted: IMGT numbering in bold; Kabat numbering underlined;
Chothia numbering italicized

The LCVR of “MC4” comprises or consists of the amino acid sequence with SEQ ID NO: 29.

SEQ ID NO: 29
EVGSYYVCPVRFQYFCEESGRSAGGGGPLGLAGSGGSDIQMTQSPSSVSASVGDRVTIT
CRASQGIYSWLAWYQQKPGKAPNLLIYTASTLQSGVPSRFSGSGSGTDFTLTISSLQPEDF
ATYYCQQANIFPLTFGGGTKVEIK
CDR sequences are highlighted: IMGT numbering in bold; Kabat numbering underlined;
Chothia numbering italicized

1. An antibody or antigen-binding fragment thereof, which specifically binds to CD40, preferably to human CD40, wherein said antibody or antigen-binding fragment thereof comprises:
(i) three light chain complementarity determining region (CDR) sequences set forth in SEQ ID NO: 1, and
(ii) three heavy chain CDR sequences set forth in SEQ ID NO: 7 or 8.

2. The antibody or antigen-binding fragment thereof according to claim 1, wherein said antibody or antigen-binding fragment comprises three heavy chain CDR sequences set forth in SEQ ID NO: 7.

3. The antibody or antigen-binding fragment thereof according to claim 1, wherein said antibody or antigen-binding fragment comprises three heavy chain CDR sequences set forth in SEQ ID NO: 8.

4. The antibody or antigen-binding fragment thereof according to any one of claims 1 to 3, wherein said antibody or antigen-binding fragment thereof comprises:
(i) a light chain variable region comprising the three following CDR sequences:
a. VL-CDR1: QGIYSW (SEQ ID NO: 9);
b. VL-CDR2: TAS;
c. V_{L}-CDR3: QQANIFPLT (SEQ ID NO: 10); and
(ii) a heavy chain variable region comprising the three following CDR sequences:
a. V_{H}-CDR1: GYTFTGX₁Y (SEQ ID NO: 11), wherein X₁ is selected from the group consisting of lysine (Lys, K) and arginine (Arg, R);
b. V_{H}-CDR2: INPDSGGT (SEQ ID NO: 12);
c. V_{H}-CDR3: ARDQPLGYCTNGVCSYFDY (SEQ ID NO: 13).

5. The antibody or antigen-binding fragment thereof according to claim 4, wherein X₁ in V_{H}-CDR1 with SEQ ID NO: 11 is lysine (Lys, K).

6. The antibody or antigen-binding fragment thereof according to claim 4, wherein X₁ in V_{H}-CDR1 with SEQ ID NO: 11 is arginine (Arg, R).

7. The antibody or antigen-binding fragment thereof according to any one of claims 4 to 6, wherein:
a) the light chain variable region further comprises the four following framework region sequences:
VL-FR1:
(SEQ ID NO: 14)
DIQMTQSPSSVSASVGDRVTITCRAS;
VL-FR2:
(SEQ ID NO: 15)
LAWYQQKPGKAPNLLIY;
VL-FR3:
(SEQ ID NO: 16)
TLQSGVPSRFSGSGSGTDFTLTISSLQPEDFATYYC;
and
VL-FR4:
(SEQ ID NO: 17)
FGGGTKVEIK;
and
b) the heavy chain variable region further comprises the four following framework region sequences:
V_{H}-FR1: QVQLVQSGAEVKKPGASVKVSCKAS (SEQ ID NO: 18);
V_{H}-FR2: MHWVRQAPGQGLEWMGW (SEQ ID NO: 19);
V_{H}-FR3: NYAQKFQGRVTMTRDTSIX₂TAYMELNRLRSDDTAVYYC (SEQ ID NO: 20), wherein X₂ is selected from the group consisting of alanine (Ala, A) and proline (Pro, P); and
V_{H}-FR4: WGQGTLVTVSS (SEQ ID NO: 21).

8. The antibody or antigen-binding fragment thereof according to any one of claims 1 to 7, wherein said antibody or antigen-binding fragment thereof comprises:
(i) a light chain variable region with SEQ ID NO: 1, or a light chain variable region sharing at least 70% of sequence identity over the non-CDR regions of SEQ ID NO: 1; and
(ii) a heavy chain variable region with SEQ ID NO: 5, 6, 7, or 8, or a heavy chain variable region sharing at least 70% of sequence identity over the non-CDR regions of SEQ ID NO: 5, 6, 7 or 8.

9. The antibody or antigen-binding fragment thereof according to any one of claims 1 to 8, wherein said antibody or antigen-binding fragment thereof comprises:
(i) a light chain variable region with SEQ ID NO: 1; and
(ii) a heavy chain variable region with an amino acid sequence selected from the group consisting of SEQ ID NOs: 5-8.

10. The antibody or antigen-binding fragment thereof according to any one of claims 1 to 9, wherein said antibody or antigen-binding fragment thereof comprises:
(i) a light chain variable region with SEQ ID NO: 1; and
(ii) a heavy chain variable region with SEQ ID NO: 5.

11. The antibody or antigen-binding fragment thereof according to any one of claims 1 to 10 wherein said antibody or antigen-binding fragment thereof is coupled to at least one masking moiety.

12. A conditionally-active antibody or antigen-binding fragment thereof, capable of specifically binding to CD40, preferably to human CD40, wherein said antibody or antigen-binding fragment thereof comprises:
(i) three light chain complementarity determining region (CDR) sequences set forth in SEQ ID NO: 1, and
(ii) three heavy chain CDR sequences set forth in SEQ ID NO: 7, 8, or 2;
and further wherein the antibody or antigen-binding fragment thereof is coupled to at least one masking moiety.

13. The conditionally-active antibody or antigen-binding fragment thereof according to claim 12, wherein the at least one masking moiety reduces or inhibits binding of the antibody or antigen-binding fragment thereof to CD40.

14. The conditionally-active antibody or antigen-binding fragment thereof according to claim 12 or 13, wherein said conditionally-active antibody or antigen-binding fragment comprises three heavy chain CDR sequences set forth in SEQ ID NO: 7.

15. The conditionally-active antibody or antigen-binding fragment thereof according to claim 12 or 13, wherein said conditionally-active antibody or antigen-binding fragment comprises three heavy chain CDR sequences set forth in SEQ ID NO: 8.

16. The conditionally-active antibody or antigen-binding fragment thereof according to claim 12 or 13, wherein said conditionally-active antibody or antigen-binding fragment comprises three heavy chain CDR sequences set forth in SEQ ID NO: 2.

17. The conditionally-active antibody or antigen-binding fragment thereof according to any one of claims 12 to 16, wherein said conditionally-active antibody or antigen-binding fragment thereof comprises:
(i) a light chain variable region with SEQ ID NO: 1, or a light chain variable region sharing at least 70% of sequence identity over the non-CDR regions of SEQ ID NO: 1; and
(ii) a heavy chain variable region with SEQ ID NO: 5, 6, 7, 8, or 2, or a heavy chain variable region sharing at least 70% of sequence identity over the non-CDR regions of SEQ ID NO: 5, 6, 7, 8, or 2.

18. The conditionally-active antibody or antigen-binding fragment thereof according to any one of claims 12 to 17, wherein said conditionally-active antibody or antigen-binding fragment thereof comprises:
(i) a light chain variable region with SEQ ID NO: 1; and
(ii) a heavy chain variable region with an amino acid sequence selected from the group consisting of SEQ ID NOs: 5, 6, 7, 8 and 2.

19. The conditionally-active antibody or antigen-binding fragment thereof according to any one of claims 12 to 18, wherein the at least one masking moiety is coupled to the N-terminus of the light chain variable region of the antibody or antigen-binding fragment thereof.

20. The conditionally-active antibody or antigen-binding fragment thereof according to any one of claims 12 to 19, further comprising at least one cleavable linker between the at least one masking moiety and the antibody or antigen-binding fragment thereof.

21. The conditionally-active antibody or antigen-binding fragment thereof according to claim 20, wherein the at least one cleavable linker is cleavable by at least one tumor-specific protease.

22. The conditionally-active antibody or antigen-binding fragment thereof according to claim 21, wherein the at least one tumor-specific protease is selected from the group consisting of matrix metalloproteinase-9 (MMP-9), urokinase-type plasminogen activator (uPa), matrix metalloproteinase-2 (MMP-2), matriptase, legumain, kallikrein-related peptidase-3, human neutrophil elastase, proteinase 3 (Pr3), cathepsin B, and cathepsin K.

23. The conditionally-active antibody or antigen-binding fragment thereof according to claim 21 or 22, wherein the at least one tumor-specific protease is MMP-9 or uPa, or a combination thereof.

24. The conditionally-active antibody or antigen-binding fragment thereof according to any one of claims 21 to 23, wherein the conditionally-active antibody or antigen-binding fragment thereof is capable of binding to CD40, preferably to human CD40, upon cleavage of the at least one cleavable linker and release of the at least one masking moiety.

25. The antibody or antigen-binding fragment thereof according to any one of claims 1 to 11, or the conditionally-active antibody or antigen-binding fragment thereof according to any one of claims 12 to 24, wherein the antibody or antigen-binding fragment thereof or the conditionally-active antibody or antigen-binding fragment thereof has pure agonist activity.

26. The antibody or antigen-binding fragment thereof, or the conditionally-active antibody or antigen-binding fragment thereof, according to claim 25, wherein pure agonist activity means that the antibody or antigen-binding fragment thereof or the conditionally-active antibody or antigen-binding fragment thereof is capable of activating the CD40 signaling pathway (i) in soluble conditions, and/or (ii) in the absence of a cross-linking reagent, and/or (iii) in an FcγR-independent manner, and/or (iv) in the absence of target-mediated crosslinking of CD40.

27. A composition comprising the antibody or antigen-binding fragment thereof, or the conditionally-active antibody or antigen-binding fragment thereof, according to any one of claims 1 to 26.

28. The composition according to claim 27, being a pharmaceutical composition and further comprising a pharmaceutically acceptable carrier or excipient.

29. A method of treating a subject in need thereof, comprising administering an effective amount of the antibody or antigen-binding fragment thereof, or of the conditionally-active antibody or antigen-binding fragment thereof, according to any one of claims 1 to 26, or of the composition according to claim 27 or 28.

30. The method according to claim 29, wherein the subject has cancer.

31. The antibody or antigen-binding fragment thereof, or the conditionally-active antibody or antigen-binding fragment thereof, according to any one of claims 1 to 26, or the composition according to claim 27 or 28, for use in treating cancer.

32. Use of the antibody or antigen-binding fragment thereof, or of the conditionally-active antibody or antigen-binding fragment thereof, according to any one of claims 1 to 26, or of the composition according to claim 27 or 28, in the manufacture of a medicament for treating cancer.

33. An isolated polynucleotide encoding the antibody or antigen-binding fragment thereof, or the conditionally-active antibody or antigen-binding fragment thereof, according to any one of claims 1 to 26.

34. A vector comprising the polynucleotide according to claim 33.

35. A host cell comprising the polynucleotide according to claim 33 or the vector according to claim 34.

36. A method of manufacturing the antibody or antigen-binding fragment thereof, or the conditionally-active antibody or antigen-binding fragment thereof, according to any one of claims 1 to 26, comprising expressing the polynucleotide according to claim 33 or the vector according to claim 34 in a cell.

37. A method of manufacturing the antibody or antigen-binding fragment thereof, or the conditionally-active antibody or antigen-binding fragment thereof, according to any one of claims 1 to 26, comprising cultivating the host cell according to claim 35 in a culture medium, and recovering the antibody or antigen-binding fragment thereof, or the conditionally-active antibody or antigen-binding fragment thereof, thereby produced by the host cell.
